SEQUENCE LISTING (32 sequences)

SEQ ID NO: 1 - protein, 12 aa, synthetic construct
GGHKAKGPRKLG

SEQ ID NO: 2 - protein, 9 aa, synthetic construct
GGHAIYPRH

SEQ ID NO: 3 - protein, 7 aa, synthetic construct
RTIGPSV

SEQ ID NO: 4 - protein, 7 aa, synthetic construct
LSSRLDA

SEQ ID NO: 5 - protein, 12 aa, synthetic construct
THRPPMWSPVWP

SEQ ID NO: 6 - protein, 12 aa, synthetic construct
FGQSSLPRDGPN

SEQ ID NO: 7 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHKAKGPRKLGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 8 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHKAKGPRKLGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 9 - protein, 734 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHAIYPRHTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 10 - protein, 724 aa, synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 11 - protein, 725 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 12 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHKAKGPRKLGTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 13 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHKAKGPRKLGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNGGHAIYPRHTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 15 - protein, 734 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHAIYPRHTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 16 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNRTIGPSVTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 17 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSRTIGPSVTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 18 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNLSSRLDATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 19 - protein, 732 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSLSSRLDATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 20 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTHRPPMWSPVWPTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 21 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTHRPPMWSPVWPTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 22 - protein, 737 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNFGQSSLPRDGPNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 23 - protein, 739 aa, synthetic construct
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSFGQSSLPRDGPNGGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 24 - protein, 734 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSGGHAIYPRHTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 25 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNRTIGPSVTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 26 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSRTIGPSVTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 27 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNLSSRLDATGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 28 - protein, 732 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSLSSRLDATTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 29 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTHRPPMWSPVWPTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 30 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTHRPPMWSPVWPTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 31 - protein, 737 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNFGQSSLPRDGPNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 32 - protein, 739 aa, synthetic construct
TAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSFGQSSLPRDGPNGGTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

(19) United States
(12) Patent Application Publication
(10) Pub. No.: US 2025/0115643 A1
(43) Pub. Date: Apr. 10, 2025

(54) RECOMBINANT AAV CAPSID PROTEINS

(71) Applicant: VectorY B.V., Amsterdam (NL)

(72) Inventors: Barbara Sanders, Amsterdam (NL); Wouter Pos, Amsterdam (NL); Pavlina Konstantinova, Amsterdam (NL); Sander van Deventer, Amsterdam (NL)

(21) Appl. No.: 18/982,262

(22) Filed: Dec. 16, 2024

Related U.S. Application Data
(63) Continuation of application No. PCT/IB2023/000364, filed on Jun. 14, 2023.
(60) Provisional application No. 63/366,369, filed on Jun. 14, 2022.

(51) Int. Cl. C07K 14/005; A61K 48/00; C12N 15/86

(52) U.S. Cl. CPC C07K 14/005; A61K 48/0025; C12N 15/86; C12N 2750/14122; C12N 2750/14143

(57) ABSTRACT

Provided herein are recombinant adeno-associated virus (AAV) capsid proteins, compositions (e.g., rAAV) comprising the capsid proteins, nucleic acids encoding the capsid proteins, and methods of making and using the capsid proteins.

### RELATED APPLICATION

This application is a continuation of International Patent Application No. PCT/IB2023/000364, filed Jun. 14, 2023, which claims the benefit of and priority to U.S. Provisional Application No. 63/366,369, filed on Jun. 14, 2022, the entire disclosure of each of which are incorporated herein by reference.

### SEQUENCE LISTING

The content of the electronically submitted Sequence Listing XML (Name: 215002_Seqlisting_ST26.XML; Size: 47,410 bytes; Created on Dec. 12, 2024) is incorporated by reference herein in its entirety.

### BACKGROUND

Adeno-associated virus (AAV) possesses unique features that makes it attractive as a vector for delivering foreign DNA into cells for the purposes of gene therapy. For example: AAV infection of cells in culture is non-cytopathic, and natural infection of humans and other animals is silent. Further, because the signals directing AAV replication, genome encapsidation and integration are contained within the inverted terminal repeats (ITRs) of the AAV genome, essentially all of the internal 4.7 kb of the AAV genome (encoding the replication and structural capsid proteins, rep-cap) can be replaced with heterologous nucleic acid sequences, such as a transgene for effecting gene therapy.

Recombinant AAV (rAAV) gene therapy vectors typically consist of a recombinant AAV genome comprising a gene therapy transgene, contained within an AAV capsid. The AAV capsid, which is formed by an assembly of 60 AAV capsid proteins, interacts with the cell surface of target cells and determines the tropism and transduction efficiency of rAAV. The blood brain barrier (BBB) remains a major obstacle for delivery of gene therapy to the central nervous system (CNS) with systemic administration. It would be highly advantageous to have rAAVs that could efficiently target cells of the CNS and cross the BBB to target the tissues in need of gene therapy.

Accordingly, there is a need in the art for tissue-specific AAV capsids for use in gene therapy applications.

### SUMMARY

Provided herein are recombinant adeno-associated virus (AAV) capsid proteins, compositions (e.g., rAAV) comprising the capsid proteins, nucleic acids encoding the capsid proteins, and methods of making and using the capsid proteins. The recombinant AAV capsid proteins provided herein are engineered to comprise a peptide within a parental AAV capsid protein. In an aspect, the recombinant AAV capsid proteins provided herein are engineered to comprise a peptide within loop IV and/or loop VIII of a parental AAV capsid protein. These recombinant AAV capsid proteins demonstrate significantly increased transduction of (motor) neurons, astrocytes, microglia, and muscle cells, as well as increased permeation of the BBB compared to the parental AAV capsid proteins. The recombinant AAV capsid proteins disclosed herein overcome previous shortcomings in the art by providing compositions and methods for delivery of viral vectors across the BBB.

In an aspect, provided herein is a recombinant adeno-associated virus (AAV) capsid protein comprising a peptide and a parental capsid protein, wherein the peptide comprises an amino acid sequence selected from the group consisting of SEQ ID NOs: 1-6. In an aspect, provided herein is a recombinant adeno-associated virus (AAV) capsid protein comprising a peptide and a parental capsid protein, wherein the peptide is comprised within loop IV and/or loop VIII of the parental capsid protein, and wherein the peptide comprises an amino acid sequence selected from the group consisting of SEQ ID NOs: 1-6. In an embodiment, the peptide is comprised within loop IV of the parental capsid protein. In an embodiment, the peptide is comprised within loop VIII of the parental capsid protein.

In an embodiment, the parental capsid protein is a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

In an embodiment, the parental capsid protein comprises an amino acid sequence that has at least 95% identity to amino acids 193-725 of SEQ ID NO: 11. In an embodiment, the parental capsid protein comprises an amino acid sequence that has at least 99% identity to amino acids 193-725 of SEQ ID NO: 11. In an embodiment, the parental capsid protein comprises the amino acid sequence of amino acids 193-725 of SEQ ID NO: 11.

In an embodiment, the parental capsid protein comprises an amino acid sequence that has at least 95% identity to amino acids 137-725 of SEQ ID NO: 11. In an embodiment, the parental capsid protein comprises an amino acid sequence that has at least 99% identity to amino acids 137-725 of SEQ ID NO: 11. In an embodiment, the parental capsid protein comprises the amino acid sequence of amino acids 137-725 of SEQ ID NO: 11.

In an embodiment, the parental capsid protein comprises an amino acid sequence that has at least 95% identity to SEQ ID NO: 10 or 11. In an embodiment, the parental capsid protein comprises an amino acid sequence that has at least 99% identity to SEQ ID NO: 10 or 11. In an embodiment, the parental capsid protein comprises the amino acid sequence of SEQ ID NO: 10 or 11.

In an embodiment, the peptide is positioned immediately C-terminal to an amino acid in the parental capsid protein corresponding to amino acid 440, 441, 442, 443, 444, 445, 446, 447, 572, 573, 574, 575, 576, 577, 578, 579, 580, 581, 582, 583, or 584 of SEQ ID NO: 11.

In an embodiment, the peptide is positioned immediately C-terminal to an amino acid in the parental capsid protein corresponding to amino acid 440 of SEQ ID NO: 11.

In an embodiment, the peptide is positioned immediately C-terminal to an amino acid in the parental capsid protein corresponding to amino acid 441 of SEQ ID NO: 11.

In an embodiment, the peptide is positioned immediately C-terminal to an amino acid in the parental capsid protein corresponding to amino acid 442 of SEQ ID NO: 11.

In an embodiment, the peptide is positioned immediately C-terminal to an amino acid in the parental capsid protein corresponding to amino acid 443 of SEQ ID NO: 11.

In an embodiment, the peptide is positioned immediately C-terminal to an amino acid in the parental capsid protein corresponding to amino acid 444 of SEQ ID NO: 11.

In an embodiment, the peptide is positioned immediately C-terminal to an amino acid in the parental capsid protein corresponding to amino acid 445 of SEQ ID NO: 11.

In an embodiment, the peptide is positioned immediately C-terminal to an amino acid in the parental capsid protein corresponding to amino acid 446 of SEQ ID NO: 11.

In an embodiment, the peptide is positioned immediately C-terminal to an amino acid in the parental capsid protein corresponding to amino acid 447 of SEQ ID NO: 11.

In an embodiment, the peptide is positioned immediately C-terminal to an amino acid in the parental capsid protein corresponding to amino acid 572 of SEQ ID NO: 11.

In an embodiment, the peptide is positioned immediately C-terminal to an amino acid in the parental capsid protein corresponding to amino acid 573 of SEQ ID NO: 11.

In an embodiment, the peptide is positioned immediately C-terminal to an amino acid in the parental capsid protein corresponding to amino acid 574 of SEQ ID NO: 11.

In an embodiment, the peptide is positioned immediately C-terminal to an amino acid in the parental capsid protein corresponding to amino acid 575 of SEQ ID NO: 11.

In an embodiment, the peptide is positioned immediately C-terminal to an amino acid in the parental capsid protein corresponding to amino acid 576 of SEQ ID NO: 11.

In an embodiment, the peptide is positioned immediately C-terminal to an amino acid in the parental capsid protein corresponding to amino acid 577 of SEQ ID NO: 11.

In an embodiment, the peptide is positioned immediately C-terminal to an amino acid in the parental capsid protein corresponding to amino acid 578 of SEQ ID NO: 11.

In an embodiment, the peptide is positioned immediately C-terminal to an amino acid in the parental capsid protein corresponding to amino acid 579 of SEQ ID NO: 11.

In an embodiment, the peptide is positioned immediately C-terminal to an amino acid in the parental capsid protein corresponding to amino acid 580 of SEQ ID NO: 11.

In an embodiment, the peptide is positioned immediately C-terminal to an amino acid in the parental capsid protein corresponding to amino acid 581 of SEQ ID NO: 11.

In an embodiment, the peptide is positioned immediately C-terminal to an amino acid in the parental capsid protein corresponding to amino acid 582 of SEQ ID NO: 11.

In an embodiment, the peptide is positioned immediately C-terminal to an amino acid in the parental capsid protein corresponding to amino acid 583 of SEQ ID NO: 11.

In an embodiment, the peptide is positioned immediately C-terminal to an amino acid in the parental capsid protein corresponding to amino acid 584 of SEQ ID NO: 11.

In an embodiment, the recombinant AAV capsid protein comprises: an amino acid sequence that has at least 95% sequence identity to amino acids 194-737 of SEQ ID NO: 12, wherein amino acids 445-456 are GGHKAKGPRKLG (SEQ ID NO: 1); an amino acid sequence that has at least 95% sequence identity to amino acids 194-737 of SEQ ID NO: 13, wherein amino acids 578-589 are GGHKAKGPRKLG (SEQ ID NO: 1); an amino acid sequence that has at least 95% sequence identity to amino acids 194-734 of SEQ ID NO: 14, wherein amino acids 445-453 are GGHAIYPRH (SEQ ID NO: 2); an amino acid sequence that has at least 95% sequence identity to amino acids 194-734 of SEQ ID NO: 15, wherein amino acids 578-586 are GGHAIYPRH (SEQ ID NO: 2); an amino acid sequence that has at least 95% sequence identity to amino acids 194-732 of SEQ ID NO: 16, wherein amino acids 445-451 are RTIGPSV (SEQ ID NO: 3); an amino acid sequence that has at least 95% sequence identity to amino acids 194-732 of SEQ ID NO: 17, wherein amino acids 578-584 are RTIGPSV (SEQ ID NO: 3); an amino acid sequence that has at least 95% sequence identity to amino acids 194-732 of SEQ ID NO: 18, wherein amino acids 445-451 are LSSRLDA (SEQ ID NO: 4); an amino acid sequence that has at least 95% sequence identity to amino acids 194-732 of SEQ ID NO: 19, wherein amino acids 578-584 are LSSRLDA (SEQ ID NO: 4); an amino acid sequence that has at least 95% sequence identity to amino acids 194-737 of SEQ ID NO: 20, wherein amino acids 445-456 are THRPPMWSPVWP (SEQ ID NO: 5); an amino acid sequence that has at least 95% sequence identity to amino acids 194-737 of SEQ ID NO: 21, wherein amino acids 578-589 are THRPPMWSPVWP (SEQ ID NO: 5); an amino acid sequence that has at least 95% sequence identity to amino acids 194-737 of SEQ ID NO: 22, wherein amino acids 445-456 are FGQSSLPRDGPN (SEQ ID NO: 6); an amino acid sequence that has at least 95% sequence identity to amino acids 194-737 of SEQ ID NO: 23, wherein amino acids 578-589 are FGQSSLPRDGPN (SEQ ID NO: 6).

In an embodiment, the recombinant AAV capsid protein comprises: an amino acid sequence that has at least 99% sequence identity to amino acids 194-737 of SEQ ID NO: 12, wherein amino acids 445-456 are GGHKAKGPRKLG (SEQ ID NO: 1); an amino acid sequence that has at least 99% sequence identity to amino acids 194-737 of SEQ ID NO: 13, wherein amino acids 578-589 are GGHKAKGPRKLG (SEQ ID NO: 1); an amino acid sequence that has at least 99% sequence identity to amino acids 194-734 of SEQ ID NO: 14, wherein amino acids 445-453 are GGHAIYPRH (SEQ ID NO: 2); an amino acid sequence that has at least 99% sequence identity to amino acids 194-734 of SEQ ID NO: 15, wherein amino acids 578-586 are GGHAIYPRH (SEQ ID NO: 2); an amino acid sequence that has at least 99% sequence identity to amino acids 194-732 of SEQ ID NO: 16, wherein amino acids 445-451 are RTIGPSV (SEQ ID NO: 3); an amino acid sequence that has at least 99% sequence identity to amino acids 194-732 of SEQ ID NO: 17, wherein amino acids 578-584 are RTIGPSV (SEQ ID NO: 3); an amino acid sequence that has at least 99% sequence identity to amino acids 194-732 of SEQ ID NO: 18, wherein amino acids 445-451 are LSSRLDA (SEQ ID NO: 4); an amino acid sequence that has at least 99% sequence identity to amino acids 194-732 of SEQ ID NO: 19, wherein amino acids 578-584 are LSSRLDA (SEQ ID NO: 4); an amino acid sequence that has at least 99% sequence identity to amino acids 194-737 of SEQ ID NO: 20, wherein amino acids 445-456 are TRPPMWSPVWP (SEQ ID NO: 5); an amino acid sequence that has at least 99% sequence identity to amino acids 194-737 of SEQ ID NO: 21, wherein amino acids 578-589 are THRPPMWSPVWP (SEQ ID NO: 5); an amino acid sequence that has at least 99% sequence identity to amino acids 194-737 of SEQ ID NO: 22, wherein amino acids 445-456 are FGQSSLPRDGPN (SEQ ID NO: 6); an amino acid sequence that has at least 99% sequence identity to amino acids 194-737 of SEQ ID NO: 23, wherein amino acids 578-589 are FGQSSLPRDGPN (SEQ ID NO: 6).

In an embodiment, the recombinant AAV capsid protein comprises: an amino acid sequence that has 100% sequence identity to amino acids 194-737 of SEQ ID NO: 12; an amino acid sequence that has 100% sequence identity to amino acids 194-737 of SEQ ID NO: 13; an amino acid sequence that has 100% sequence identity to amino acids 194-734 of SEQ ID NO: 14; an amino acid sequence that has 100% sequence identity to amino acids 194-734 of SEQ ID NO: 15; an amino acid sequence that has 100% sequence identity to amino acids 194-732 of SEQ ID NO: 16; an amino acid sequence that has 100% sequence identity to amino acids 194-732 of SEQ ID NO: 17; an amino acid sequence that has 100% sequence identity to amino acids 194-732 of SEQ ID NO: 18; an amino acid sequence that has 100% sequence identity to amino acids 194-732 of SEQ ID NO: 19; an amino acid sequence that has 100% sequence identity to amino acids 194-737 of SEQ ID NO: 20; an amino acid sequence that has 100% sequence identity to amino acids 194-737 of SEQ ID NO: 21; an amino acid sequence that has 100% sequence identity to amino acids 194-737 of SEQ ID NO: 22; an amino acid sequence that has 100% sequence identity to amino acids 194-737 of SEQ ID NO: 23.

In an embodiment, the recombinant AAV capsid protein comprises: an amino acid sequence that has at least 95% sequence identity to amino acids 138-737 of SEQ ID NO: 12, wherein amino acids 445-456 are GGHKAKGPRKLG (SEQ ID NO: 1); an amino acid sequence that has at least 95% sequence identity to amino acids 138-737 of SEQ ID NO: 13, wherein amino acids 578-589 are GGHKAKGPRKLG (SEQ ID NO: 1); an amino acid sequence that has at least 95% sequence identity to amino acids 138-734 of SEQ ID NO: 14, wherein amino acids 445-453 are GGHAIYPRH (SEQ ID NO: 2); an amino acid sequence that has at least 95% sequence identity to amino acids 138-734 of SEQ ID NO: 15, wherein amino acids 578-586 are GGHAIYPRH (SEQ ID NO: 2); an amino acid sequence that has at least 95% sequence identity to amino acids 138-732 of SEQ ID NO: 16, wherein amino acids 445-451 are RTIGPSV (SEQ ID NO: 3); an amino acid sequence that has at least 95% sequence identity to amino acids 138-732 of SEQ ID NO: 17, wherein amino acids 578-584 are RTIGPSV (SEQ ID NO: 3); an amino acid sequence that has at least 95% sequence identity to amino acids 138-732 of SEQ ID NO: 18, wherein amino acids 445-451 are LSSRLDA (SEQ ID NO: 4); an amino acid sequence that has at least 95% sequence identity to amino acids 138-732 of SEQ ID NO: 19, wherein amino acids 578-584 are LSSRLDA (SEQ ID NO: 4); an amino acid sequence that has at least 95% sequence identity to amino acids 138-737 of SEQ ID NO: 20, wherein amino acids 445-456 are THRPPMWSPVWP (SEQ ID NO: 5); an amino acid sequence that has at least 95% sequence identity to amino acids 138-737 of SEQ ID NO: 21, wherein amino acids 578-589 are THRPPMWSPVWP (SEQ ID NO: 5); an amino acid sequence that has at least 95% sequence identity to amino acids 138-737 of SEQ ID NO: 22, wherein amino acids 445-456 are FGQSSLPRDGPN (SEQ ID NO: 6); an amino acid sequence that has at least 95% sequence identity to amino acids 138-737 of SEQ ID NO: 23, wherein amino acids 578-589 are FGQSSLPRDGPN (SEQ ID NO: 6).

In an embodiment, the recombinant AAV capsid protein comprises: an amino acid sequence that has at least 99% sequence identity to amino acids 138-737 of SEQ ID NO: 12, wherein amino acids 445-456 are GGHKAKGPRKLG (SEQ ID NO: 1); an amino acid sequence that has at least 99% sequence identity to amino acids 138-737 of SEQ ID NO: 13, wherein amino acids 578-589 are GGHKAKGPRKLG (SEQ ID NO: 1); an amino acid sequence that has at least 99% sequence identity to amino acids 138-734 of SEQ ID NO: 14, wherein amino acids 445-453 are GGHAIYPRH (SEQ ID NO: 2); an amino acid sequence that has at least 99% sequence identity to amino acids 138-734 of SEQ ID NO: 15, wherein amino acids 578-586 are GGHAIYPRH (SEQ ID NO: 2); an amino acid sequence that has at least 99% sequence identity to amino acids 138-732 of SEQ ID NO: 16, wherein amino acids 445-451 are RTIGPSV (SEQ ID NO: 3); an amino acid sequence that has at least 99% sequence identity to amino acids 138-732 of SEQ ID NO: 17, wherein amino acids 578-584 are RTIGPSV (SEQ ID NO: 3); an amino acid sequence that has at least 99% sequence identity to amino acids 138-732 of SEQ ID NO: 18, wherein amino acids 445-451 are LSSRLDA (SEQ ID NO: 4); an amino acid sequence that has at least 99% sequence identity to amino acids 138-732 of SEQ ID NO: 19, wherein amino acids 578-584 are LSSRLDA (SEQ ID NO: 4); an amino acid sequence that has at least 99% sequence identity to amino acids 138-737 of SEQ ID NO: 20, wherein amino acids 445-456 are THRPPMWSPVWP (SEQ ID NO: 5); an amino acid sequence that has at least 99% sequence identity to amino acids 138-737 of SEQ ID NO: 21, wherein amino acids 578-589 are THRPPMWSPVWP (SEQ ID NO: 5); an amino acid sequence that has at least 99% sequence identity to amino acids 138-737 of SEQ ID NO: 22, wherein amino acids 445-456 are FGQSSLPRDGPN (SEQ ID NO: 6); an amino acid sequence that has at least 99% sequence identity to amino acids 138-737 of SEQ ID NO: 23, wherein amino acids 578-589 are FGQSSLPRDGPN (SEQ ID NO: 6).

In an embodiment, the recombinant AAV capsid protein comprises: an amino acid sequence that has 100% sequence identity to amino acids 138-737 of SEQ ID NO: 12; an amino acid sequence that has 100% sequence identity to amino acids 138-737 of SEQ ID NO: 13; an amino acid sequence that has 100% sequence identity to amino acids 138-734 of SEQ ID NO: 14; an amino acid sequence that has 100% sequence identity to amino acids 138-734 of SEQ ID NO: 15; an amino acid sequence that has 100% sequence identity to amino acids 138-732 of SEQ ID NO: 16; an amino acid sequence that has 100% sequence identity to amino acids 138-732 of SEQ ID NO: 17; an amino acid sequence that has 100% sequence identity to amino acids 138-732 of SEQ ID NO: 18; an amino acid sequence that has 100% sequence identity to amino acids 138-732 of SEQ ID NO: 19; an amino acid sequence that has 100% sequence identity to amino acids 138-737 of SEQ ID NO: 20; an amino acid sequence that has 100% sequence identity to amino acids 138-737 of SEQ ID NO: 21; an amino acid sequence that has 100% sequence identity to amino acids 138-737 of SEQ ID NO: 22; an amino acid sequence that has 100% sequence identity to amino acids 138-737 of SEQ ID NO: 23.

In an embodiment, the recombinant AAV capsid protein comprises: an amino acid sequence that has at least 95% sequence identity to amino acids 1-737 of SEQ ID NO: 12, wherein amino acids 445-456 are GGHKAKGPRKLG (SEQ ID NO: 1); an amino acid sequence that has at least 95% sequence identity to amino acids 1-737 of SEQ ID NO: 13, wherein amino acids 578-589 are GGHKAKGPRKLG (SEQ ID NO: 1); an amino acid sequence that has at least 95% sequence identity to amino acids 1-734 of SEQ ID NO: 14, wherein amino acids 445-453 are GGHAIYPRH (SEQ ID NO: 2); an amino acid sequence that has at least 95% sequence identity to amino acids 1-734 of SEQ ID NO: 15, wherein amino acids 578-586 are GGHAIYPRH (SEQ ID NO: 2); an amino acid sequence that has at least 95% sequence identity to amino acids 1-732 of SEQ ID NO: 16, wherein amino acids 445-451 are RTIGPSV (SEQ ID NO: 3); an amino acid sequence that has at least 95% sequence identity to amino acids 1-732 of SEQ ID NO: 17, wherein amino acids 578-584 are RTIGPSV (SEQ ID NO: 3); an amino acid sequence that has at least 95% sequence identity to amino acids 1-732 of SEQ ID NO: 18, wherein amino acids 445-451 are LSSRLDA (SEQ ID NO: 4); an amino acid sequence that has at least 95% sequence identity to amino acids 1-732 of SEQ ID NO: 19, wherein amino acids 578-584 are LSSRLDA (SEQ ID NO: 4); an amino acid sequence that has at least 95% sequence identity to amino acids 1-737 of SEQ ID NO: 20, wherein amino acids 445-456 are THRPPMWSPVWP (SEQ ID NO: 5); an amino acid sequence that has at least 95% sequence identity to amino acids 1-737 of SEQ ID NO: 21, wherein amino acids 578-589 are THRPPMWSPVWP (SEQ ID NO: 5); an amino acid sequence that has at least 95% sequence identity to amino acids 1-737 of SEQ ID NO: 22, wherein amino acids 445-456 are FGQSSLPRDGPN (SEQ ID NO: 6); an amino acid sequence that has at least 95% sequence identity to amino acids 1-737 of SEQ ID NO: 23, wherein amino acids 578-589 are FGQSSLPRDGPN (SEQ ID NO: 6).

In an embodiment, the recombinant AAV capsid protein comprises: an amino acid sequence that has at least 99% sequence identity to amino acids 1-737 of SEQ ID NO: 12, wherein amino acids 445-456 are GGHKAKGPRKLG (SEQ ID NO: 1); an amino acid sequence that has at least 99% sequence identity to amino acids 1-737 of SEQ ID NO: 13, wherein amino acids 578-589 are GGHKAKGPRKLG (SEQ ID NO: 1); an amino acid sequence that has at least 99% sequence identity to amino acids 1-734 of SEQ ID NO: 14, wherein amino acids 445-453 are GGHAIYPRH (SEQ ID NO: 2); an amino acid sequence that has at least 99% sequence identity to amino acids 1-734 of SEQ ID NO: 15, wherein amino acids 578-586 are GGHAIYPRH (SEQ ID NO: 2); an amino acid sequence that has at least 99% sequence identity to amino acids 1-732 of SEQ ID NO: 16, wherein amino acids 445-451 are RTIGPSV (SEQ ID NO: 3); an amino acid sequence that has at least 99% sequence identity to amino acids 1-732 of SEQ ID NO: 17, wherein amino acids 578-584 are RTIGPSV (SEQ ID NO: 3); an amino acid sequence that has at least 99% sequence identity to amino acids 1-732 of SEQ ID NO: 18, wherein amino acids 445-451 are LSSRLDA (SEQ ID NO: 4); an amino acid sequence that has at least 99% sequence identity to amino acids 1-732 of SEQ ID NO: 19, wherein amino acids 578-584 are LSSRLDA (SEQ ID NO: 4); an amino acid sequence that has at least 99% sequence identity to amino acids 1-737 of SEQ ID NO: 20, wherein amino acids 445-456 are THRPPMWSPVWP (SEQ ID NO: 5); an amino acid sequence that has at least 99% sequence identity to amino acids 1-737 of SEQ ID NO: 21, wherein amino acids 578-589 are THRPPMWSPVWP (SEQ ID NO: 5); an amino acid sequence that has at least 99% sequence identity to amino acids 1-737 of SEQ ID NO: 22, wherein amino acids 445-456 are FGQSSLPRDGPN (SEQ ID NO: 6); an amino acid sequence that has at least 99% sequence identity to amino acids 1-737 of SEQ ID NO: 23, wherein amino acids 578-589 are FGQSSLPRDGPN (SEQ ID NO: 6).

In an embodiment, the recombinant AAV capsid protein comprises an amino acid sequence selected from the group consisting of SEQ ID NOs: 12-23. In an embodiment, the amino acid sequence of the AAV capsid protein consists of an amino acid sequence selected from the group consisting of SEQ ID NOs: 12-23.

In an aspect, provided herein is an isolated polynucleotide encoding a recombinant AAV capsid protein disclosed herein.

In an aspect, provided herein is a vector comprising a polynucleotide disclosed herein.

In an embodiment, the vector is a plasmid or a viral vector. In an embodiment, the viral vector is a retrovirus vector, a herpes virus vector, a baculovirus vector, or an adenovirus vector. In an embodiment, the vector is an expression vector.

In an aspect, provided herein is a recombinant cell comprising a polynucleotide or a vector disclosed herein.

In an aspect, provided herein is a method of producing an AAV capsid protein, the method comprising culturing a recombinant cell disclosed herein under conditions where the polynucleotide is expressed, and the capsid protein is produced.

In an aspect, provided herein is a recombinant adeno-associated virus (rAAV) comprising a capsid comprising one or more recombinant capsid protein disclosed herein; and an rAAV genome.

In an embodiment, the rAAV genome comprises a transgene. In an embodiment, the transgene encodes a polypeptide or non-coding RNA. In an embodiment, the transgene encodes an antibody or a fragment thereof. In an embodiment, the transgene encodes an scFv, nanobody, or VHH.

In an aspect, provided herein is a method of delivering a transgene to a cell, the method comprising contacting the cell with an rAAV disclosed herein, under conditions where the cell is transduced, and the transgene is expressed. In an embodiment, the cell is a muscle cell, microglia, astrocyte, neuron, blood brain barrier endothelial cell (BBB EC), pericyte, oligodendrocyte, ependymal cell, or cardiomyocyte. In an embodiment, the neuron is a pyramidal neuron, rosehip neuron, or purkinje cell.

In an aspect, provided herein is a method of delivering a transgene to brain, spinal cord, or muscle tissue in a subject in need thereof, the method comprising administering an rAAV disclosed herein. In an embodiment, the subject has a disease associated with brain, spinal cord, or muscle tissue.

In an aspect, provided herein is a method of treating a neuromuscular or neurodegenerative disease or disorder in a subject in need thereof, comprising administering an effective amount of an rAAV disclosed herein to the subject.

In an embodiment, the neuromuscular disease or disorder is selected from the group consisting of myopathy, hereditary cardiomyopathy, metabolic myopathy, distal myopathy, muscular dystrophy, congenital myopathy, spinal muscular atrophy (SMA), motor neuron disease, congenital myopathy, congenital muscular dystrophy, motor neuron disease, Duchenne muscular dystrophy, Becker muscular dystrophy, limb-girdle muscular dystrophies, myotonic dystrophy, myotubular myopathy, centronuclear myopathy, nemaline myopathy, selenoprotein N-related myopathy, Pompe disease, glycogen storage disease III, spinal muscular atrophy, amyotrophic lateral sclerosis (ALS), Charcot-Marie-Tooth disease, multiple sclerosis, myositis, polymyositis, and dermatomyositis.

In an embodiment, the neurodegenerative disease or disorder is selected from the group consisting of motor neuron disease (MND), parkinsonism syndrome, Alzheimer's dementia, progressive supranuclear palsy (PSP), Huntington's disease, multiple system atrophy (MSA), spinocerebellar ataxia (SCA), and Friedreich's ataxia.

In an embodiment, the rAAV is administered to the subject intravenously, intraperitoneally, subcutaneously, intramuscularly, intrathecally, or intradermally.

### BRIEF DESCRIPTION OF THE DRAWINGS

FIG. 1A is a graph showing GFP expression in myotubes following transduction with recombinant AAV capsid proteins that comprise VYPep2, VYPep4, VYPep5, or VYPep11 in loop IV or loop VIII compared to parental capsid proteins with no peptide insertion. FIG. 1B is a graph showing GFP protein expression in human myotubes following transduction with recombinant AAV capsid proteins that comprise VYPep4 or VYPep5 compared to the parental capsid protein with no peptide insertion.

FIG. 2A is a graph showing the percent positive GFP human astrocytes following transduction with recombinant AAV capsid proteins that comprise VYPep4 or VYPep5 in loop IV or loop VIII compared to parental capsid proteins with no peptide insertion, at an MOI of 1×10⁶. FIG. 2B is a graph showing the percent positive GFP human astrocytes following transduction with recombinant AAV capsid proteins that comprise VYPep4 or VYPep5 in loop IV or loop VIII compared to parental capsid proteins with no peptide insertion, at an MOI of 1×10⁴.

FIG. 3 is a graph showing the percent positive GFP cells on the brain side of the blood brain barrier following transduction of astrocytes with recombinant AAV capsid proteins comprising VYPep4 compared to parental capsid proteins with no peptide insertion, in a blood brain barrier microfluid model.

FIG. 4 is a graph showing the percent positive GFP non-human primate (NHP) astrocytes following transduction with recombinant AAV capsid proteins comprising VYPep4 in loop IV compared to parental capsid proteins with no peptide insertion, at an MOI of 1×10⁵ and 1×10⁶.

FIG. 5 is a graph showing the percent positive GFP human motor neurons following transduction with recombinant AAV capsid proteins comprising VYPep4 in loop IV compared to parental capsid proteins with no peptide insertion, at an MOI of 1×10⁵ and 1×10⁶.

### DETAILED DESCRIPTION

Provided herein are recombinant adeno-associated virus (AAV) capsid proteins, compositions (e.g., rAAV) comprising the capsid proteins, nucleic acids encoding the capsid proteins, and methods of making and using the capsid proteins. The recombinant AAV capsid proteins provided herein are engineered to comprise a peptide within a parental AAV capsid protein. In an aspect, the recombinant AAV capsid proteins provided herein are engineered to comprise a peptide within loop IV and/or loop VIII of a parental AAV capsid protein. These recombinant AAV capsid proteins demonstrate significantly increased permeation of the blood brain barrier (BBB) compared to the parental AAV capsid proteins.

### Definitions

As used herein, the term “AAV” is a standard abbreviation for adeno-associated virus.

As used herein, the term “parental capsid protein” refers to an AAV capsid protein into which a peptide is inserted. A parental capsid protein can be a naturally occurring AAV capsid protein (e.g., of any clade or clone described in Bello et al., Sci. Rep. (2014) 4(6644): 1-11. doi: 10.1038/srep06644 or Gao et al., J Virol. (June 2004); 78(12): 6381-6388. doi: 10.1128/JVI.78.12.6381-6388.2004) or a non-naturally occurring AAV capsid protein. Suitable non-naturally occurring AAV capsid proteins include, without limitation, chimeric capsid proteins and capsid proteins engineered to contain one or more amino acid insertion, deletion, or substitution relative to a naturally occurring AAV capsid protein.

As used herein, the term “recombinant adeno-associated virus” or “rAAV” refers to an AAV comprising a genome lacking functional rep and cap genes.

As used herein, the term “cap gene” refers to a nucleic acid sequence that encodes a capsid protein. For AAV, the capsid protein may be VP1, VP2, or VP3. VP1, VP2, and/or VP3 capsid proteins assemble into a capsid that surrounds the rAAV genome.

As used herein, the term “rep gene” refers to the nucleic acid sequences that encode the non-structural proteins (e.g., rep78, rep68, rep52, and rep40) required for the replication and production of an AAV.

As used herein, the term “loop IV” refers to the amino acids in an AAV capsid protein corresponding to amino acids 434-466 SEQ ID NO: 10 or 435-467 of SEQ ID NO: 11, or the corresponding amino acid of a different parental capsid protein.

As used herein, the term “loop VIII” refers to the amino acids in an AAV capsid protein corresponding to amino acids 563-592 SEQ ID NO: 10 or 564-593 of SEQ ID NO: 11, or the corresponding amino acid of a different parental capsid protein.

As used herein, the term “rAAV genome” refers to a nucleic acid molecule (e.g., DNA and/or RNA) comprising the genome sequence of an rAAV. The skilled artisan will appreciate that where an rAAV genome comprises a transgene (e.g., an antibody heavy chain or light chain coding sequence operably linked to a transcriptional regulatory element), the rAAV genome can be in the sense or antisense orientation relative to the direction of transcription of the transgene.

As used herein, an “isolated polynucleotide” refers to a polynucleotide that has been separated from one or more nucleic acid molecules present in the natural source of the polynucleotide.

As used herein, a “vector” refers to a nucleic acid molecule that is a vehicle for introducing a nucleic acid molecule (e.g., a polynucleotide disclosed herein) into a cell.

As used herein, an “expression vector” refers to a vector comprising transcriptional regulatory elements operably linked to a gene of interest (e.g., a polynucleotide disclosed herein) that facilitate the expression of the gene of interest in a cell and/or a cell free expression system.

As used herein, the term “transgene” refers to a non-AAV nucleic acid sequence that encodes a polypeptide (e.g., an antibody or scFv) or non-coding RNA (e.g., an miRNA, shRNA, siRNA, antisense RNA, gRNA, antagomir, miRNA sponge, RNA aptazyme, RNA ribozyme, or RNA aptamer).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a polynucleotide sequence (e.g., a transgene disclosed herein) to be transcribed. Typically, gene expression is placed under the control of a TRE comprising one or more promoter and/or enhancer elements. The transgene is “operably linked” to the TRE if the transcription of the transgene is controlled or influenced by the TRE. The promoter and enhancer elements of the TRE may be in any orientation and/or distance from the transgene, as long as the desired transcriptional activity is obtained. In an embodiment, the TRE is upstream from the transgene.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. When a sequence is described herein as being a certain percentage identical to a reference sequence, the percentage identity to the reference sequence is determined across the full length of the reference sequence.

As used herein, the term “effective amount” in the context of the administration of an AAV to a subject refers to the amount of the AAV that achieves a desired prophylactic or therapeutic effect.

### AAV Capsid Proteins

In an aspect, provided herein is a recombinant adeno-associated virus (AAV) capsid protein comprising a peptide and a parental AAV capsid protein. The amino acid sequences of exemplary peptides used in the recombinant AAV capsid proteins disclosed herein are provided in Table 1 below.

TABLE 1
Amino acid sequences of exemplary peptides

| | SEQ | |
|---|---|---|
| Description | ID NO | Amino acid sequence |
| VYPep2 | 1 | GGHKAKGPRKLG |
| VYPep3 | 2 | GGHAIYPRH |
| VYPep4 | 3 | RTIGPSV |
| VYPep5 | 4 | LSSRLDA |
| VYPep6 | 5 | THRPPMWSPVWP |
| VYPep11 | 6 | FGQSSLPRDGPN |

In an aspect, provided herein is a recombinant adeno-associated virus (AAV) capsid protein comprising a peptide and a parental capsid protein, wherein the peptide comprises an amino acid sequence selected from the group consisting of SEQ ID NOs: 1-6. In an aspect, provided herein is a recombinant adeno-associated virus (AAV) capsid protein comprising a peptide and a parental capsid protein, wherein the peptide is comprised within loop IV and/or loop VIII of the parental capsid protein, and wherein the peptide comprises an amino acid sequence selected from the group consisting of SEQ ID NOs: 1-6.

In an embodiment, the peptide comprises the amino acid sequence of SEQ ID NO: 1, and the peptide is comprised within loop IV of the parental capsid protein. In an embodiment, the peptide comprises the amino acid sequence of SEQ ID NO: 2, and the peptide is comprised within loop IV of the parental capsid protein. In an embodiment, the peptide comprises the amino acid sequence of SEQ ID NO: 3, and the peptide is comprised within loop IV of the parental capsid protein. In an embodiment, the peptide comprises the amino acid sequence of SEQ ID NO: 4, and the peptide is comprised within loop IV of the parental capsid protein. In an embodiment, the peptide comprises the amino acid sequence of SEQ ID NO: 5, and the peptide is comprised within loop IV of the parental capsid protein. In an embodiment, the peptide comprises the amino acid sequence of SEQ ID NO: 6, and the peptide is comprised within loop IV of the parental capsid protein.

In an embodiment, the peptide comprises the amino acid sequence of SEQ ID NO: 1, and the peptide is comprised within loop VIII of the parental capsid protein. In an embodiment, the peptide comprises the amino acid sequence of SEQ ID NO: 2, and the peptide is comprised within loop VIII of the parental capsid protein. In an embodiment, the peptide comprises the amino acid sequence of SEQ ID NO: 3, and the peptide is comprised within loop VIII of the parental capsid protein. In an embodiment, the peptide comprises the amino acid sequence of SEQ ID NO: 4, and the peptide is comprised within loop VIII of the parental capsid protein. In an embodiment, the peptide comprises the amino acid sequence of SEQ ID NO: 5, and the peptide is comprised within loop VIII of the parental capsid protein. In an embodiment, the peptide comprises the amino acid sequence of SEQ ID NO: 6, and the peptide is comprised within loop VIII of the parental capsid protein.

In an embodiment, the recombinant AAV capsid protein comprises a first peptide within loop IV of the parental capsid protein and a second peptide within loop VIII of the parental capsid protein. In an embodiment, the first peptide and the second peptide are the same peptide. In an embodiment, the first peptide and the second peptide are different peptides. In an embodiment, the first peptide comprises the amino acid sequence of SEQ ID NO: 1 and the second peptide comprises an amino acid sequence selected from the group consisting of SEQ ID NOs: 1-6. In an embodiment, the first peptide comprises the amino acid sequence of SEQ ID NO: 2 and the second peptide comprises an amino acid sequence selected from the group consisting of SEQ ID NOs: 1-6. In an embodiment, the first peptide comprises the amino acid sequence of SEQ ID NO: 3 and the second peptide comprises an amino acid sequence selected from the group consisting of SEQ ID NOs: 1-6. In an embodiment, the first peptide comprises the amino acid sequence of SEQ ID NO: 4 and the second peptide comprises an amino acid sequence selected from the group consisting of SEQ ID NOs: 1-6. In an embodiment, the first peptide comprises the amino acid sequence of SEQ ID NO: 5 and the second peptide comprises an amino acid sequence selected from the group consisting of SEQ ID NOs: 1-6. In an embodiment, the first peptide comprises the amino acid sequence of SEQ ID NO: 6 and the second peptide comprises an amino acid sequence selected from the group consisting of SEQ ID NOs: 1-6.

In an embodiment, the parental capsid protein is a VP1 capsid protein. In an embodiment, the parental capsid protein is a VP2 capsid protein. In an embodiment, the parental capsid protein is a VP3 capsid protein.

In an embodiment, the parental capsid protein is a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, AAV5.2, or AAV5 capsid protein, or an engineered variant thereof.

In an embodiment, the parental capsid protein is an engineered variant capsid protein comprising amino acid sequences from at least two different AAV capsid proteins. In an embodiment, the parental capsid protein comprises amino acid sequences from an AAV2 capsid protein and an AAV5 capsid protein. In an embodiment, the parental capsid protein comprises a VP1 amino acid sequence from AAV2 and a VP2 and VP3 amino acid sequence from AAV5.

In an embodiment, the parental capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to amino acids 193-725 of SEQ ID NO: 11. In an embodiment, the parental capsid protein comprises an amino acid sequence that has at least 99% identity amino acids 193-725 of SEQ ID NO: 11. In an embodiment, the parental capsid protein comprises an amino acid sequence that has 100% identity to amino acids 193-725 of SEQ ID NO: 11.

In an embodiment, the parental capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to amino acids 137-725 of SEQ ID NO: 11. In an embodiment, the parental capsid protein comprises an amino acid sequence that has at least 99% identity to amino acids 137-725 of SEQ ID NO: 11. In an embodiment, the parental capsid protein comprises an amino acid sequence that has 100% identity to amino acids 137-725 of SEQ ID NO: 11.

In an embodiment, the parental capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% identity to the amino acid sequence of SEQ ID NO: 10 or 11. In an embodiment, the parental capsid protein comprises an amino acid sequence that has at least 99% identity to the amino acid sequence of SEQ ID NO: 10 or 11. In an embodiment, the parental capsid protein comprises an amino acid sequence that has 100% identity to the amino acid sequence of SEQ ID NO: 10 or 11.

In an embodiment, the peptide is positioned immediately C-terminal to an amino acid in the parental capsid corresponding to amino acid 440, 441, 442, 443, 444, 445, 446, 447, 572, 573, 574, 575, 576, 577, 578, 579, 580, 581, 582, 583, or 584 of SEQ ID NO: 11.

In an embodiment, the peptide is positioned immediately C-terminal to an amino acid in the parental capsid corresponding to amino acid 440 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 440 of SEQ ID NO: 11. In an embodiment, the peptide comprises the amino acid sequence of SEQ ID NO: 2 and the peptide is positioned immediately C-terminal to an amino acid in the parental capsid corresponding to amino acid 440 of SEQ ID NO: 11. In an embodiment, the peptide comprises the amino acid sequence of SEQ ID NO: 3 and the peptide is positioned immediately C-terminal to an amino acid in the parental capsid corresponding to amino acid 440 of SEQ ID NO: 11. In an embodiment, the peptide comprises the amino acid sequence of SEQ ID NO: 4 and the peptide is positioned immediately C-terminal to an amino acid in the parental capsid corresponding to amino acid 440 of SEQ ID NO: 11. In an embodiment, the peptide comprises the amino acid sequence of SEQ ID NO: 5 and the peptide is positioned immediately C-terminal to an amino acid in the parental capsid corresponding to amino acid 440 of SEQ ID NO: 11. In an embodiment, the peptide comprises the amino acid sequence of SEQ ID NO: 6 and the peptide is positioned immediately C-terminal to an amino acid in the parental capsid corresponding to amino acid 440 of SEQ ID NO: 11.

In an embodiment, the peptide is positioned immediately C-terminal to an amino acid in the parental capsid corresponding to amino acid 441 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 441 of SEQ ID NO: 11. In an embodiment, the peptide comprises the amino acid sequence of SEQ ID NO: 2 and the peptide is positioned immediately C-terminal to an amino acid in the parental capsid corresponding to amino acid 441 of SEQ ID NO: 11. In an embodiment, the peptide comprises the amino acid sequence of SEQ ID NO: 3 and the peptide is positioned immediately C-terminal to an amino acid in the parental capsid corresponding to amino acid 441 of SEQ ID NO: 11. In an embodiment, the peptide comprises the amino acid sequence of SEQ ID NO: 4 and the peptide is positioned immediately C-terminal to an amino acid in the parental capsid corresponding to amino acid 441 of SEQ ID NO: 11. In an embodiment, the peptide comprises the amino acid sequence of SEQ ID NO: 5 and the peptide is positioned immediately C-terminal to an amino acid in the parental capsid corresponding to amino acid 441 of SEQ ID NO: 11. In an embodiment, the peptide comprises the amino acid sequence of SEQ ID NO: 6 and the peptide is positioned immediately C-terminal to an amino acid in the parental capsid corresponding to amino acid 441 of SEQ ID NO: 11.

In an embodiment, the peptide is positioned immediately C-terminal to an amino acid in the parental capsid corresponding to amino acid 442 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 442 of SEQ ID NO: 11. In an embodiment, the peptide comprises the amino acid sequence of SEQ ID NO: 2 and the peptide is positioned immediately C-terminal to an amino acid in the parental capsid corresponding to amino acid 442 of SEQ ID NO: 11. In an embodiment, the peptide comprises the amino acid sequence of SEQ ID NO: 3 and the peptide is positioned immediately C-terminal to an amino acid in the parental capsid corresponding to amino acid 442 of SEQ ID NO: 11. In an embodiment, the peptide comprises the amino acid sequence of SEQ ID NO: 4 and the peptide is positioned immediately C-terminal to an amino acid in the parental capsid corresponding to amino acid 442 of SEQ ID NO: 11. In an embodiment, the peptide comprises the amino acid sequence of SEQ ID NO: 5 and the peptide is positioned immediately C-terminal to an amino acid in the parental capsid corresponding to amino acid 442 of SEQ ID NO: 11. In an embodiment, the peptide comprises the amino acid sequence of SEQ ID NO: 6 and the peptide is positioned immediately C-terminal to an amino acid in the parental capsid corresponding to amino acid 442 of SEQ ID NO: 11.

In an embodiment, the peptide is positioned immediately C-terminal to an amino acid in the parental capsid corresponding to amino acid 443 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 443 of SEQ ID NO: 11. In an embodiment, the peptide comprises the amino acid sequence of SEQ ID NO: 2 and the peptide is positioned immediately C-terminal to an amino acid in the parental capsid corresponding to amino acid 443 of SEQ ID NO: 11. In an embodiment, the peptide comprises the amino acid sequence of SEQ ID NO: 3 and the peptide is positioned immediately C-terminal to an amino acid in the parental capsid corresponding to amino acid 443 of SEQ ID NO: 11. In an embodiment, the peptide comprises the amino acid sequence of SEQ ID NO: 4 and the peptide is positioned immediately C-terminal to an amino acid in the parental capsid corresponding to amino acid 443 of SEQ ID NO: 11. In an embodiment, the peptide comprises the amino acid sequence of SEQ ID NO: 5 and the peptide is positioned immediately C-terminal to an amino acid in the parental capsid corresponding to amino acid 443 of SEQ ID NO: 11. In an embodiment, the peptide comprises the amino acid sequence of SEQ ID NO: 6 and the peptide is positioned immediately C-terminal to an amino acid in the parental capsid corresponding to amino acid 443 of SEQ ID NO: 11.

In an embodiment, the peptide is positioned immediately C-terminal to an amino acid in the parental capsid corresponding to amino acid 444 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 444 of SEQ ID NO: 11. In an embodiment, the peptide comprises the amino acid sequence of SEQ ID NO: 2 and the peptide is positioned immediately C-terminal to an amino acid in the parental capsid corresponding to amino acid 444 of SEQ ID NO: 11. In an embodiment, the peptide comprises the amino acid sequence of SEQ ID NO: 3 and the peptide is positioned immediately C-terminal to an amino acid in the parental capsid corresponding to amino acid 444 of SEQ ID NO: 11. In an embodiment, the peptide comprises the amino acid sequence of SEQ ID NO: 4 and the peptide is positioned immediately C-terminal to an amino acid in the parental capsid corresponding to amino acid 444 of SEQ ID NO: 11. In an embodiment, the peptide comprises the amino acid sequence of SEQ ID NO: 5 and the peptide is positioned immediately C-terminal to an amino acid in the parental capsid corresponding to amino acid 444 of SEQ ID NO: 11. In an embodiment, the peptide comprises the amino acid sequence of SEQ ID NO: 6 and the peptide is positioned immediately C-terminal to an amino acid in the parental capsid corresponding to amino acid 444 of SEQ ID NO: 11.

In an embodiment, the peptide is positioned immediately C-terminal to an amino acid in the parental capsid corresponding to amino acid 445 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 445 of SEQ ID NO: 11. In an embodiment, the peptide comprises the amino acid sequence of SEQ ID NO: 2 and the peptide is positioned immediately C-terminal to an amino acid in the parental capsid corresponding to amino acid 445 of SEQ ID NO: 11. In an embodiment, the peptide comprises the amino acid sequence of SEQ ID NO: 3 and the peptide is positioned immediately C-terminal to an amino acid in the parental capsid corresponding to amino acid 445 of SEQ ID NO: 11. In an embodiment, the peptide comprises the amino acid sequence of SEQ ID NO: 4 and the peptide is positioned immediately C-terminal to an amino acid in the parental capsid corresponding to amino acid 445 of SEQ ID NO: 11. In an embodiment, the peptide comprises the amino acid sequence of SEQ ID NO: 5 and the peptide is positioned immediately C-terminal to an amino acid in the parental capsid corresponding to amino acid 445 of SEQ ID NO: 11. In an embodiment, the peptide comprises the amino acid sequence of SEQ ID NO: 6 and the peptide is positioned immediately C-terminal to an amino acid in the parental capsid corresponding to amino acid 445 of SEQ ID NO: 11.

In an embodiment, the peptide is positioned immediately C-terminal to an amino acid in the parental capsid corresponding to amino acid 446 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 446 of SEQ ID NO: 11. In an embodiment, the peptide comprises the amino acid sequence of SEQ ID NO: 2 and the peptide is positioned immediately C-terminal to an amino acid in the parental capsid corresponding to amino acid 446 of SEQ ID NO: 11. In an embodiment, the peptide comprises the amino acid sequence of SEQ ID NO: 3 and the peptide is positioned immediately C-terminal to an amino acid in the parental capsid corresponding to amino acid 446 of SEQ ID NO: 11. In an embodiment, the peptide comprises the amino acid sequence of SEQ ID NO: 4 and the peptide is positioned immediately C-terminal to an amino acid in the parental capsid corresponding to amino acid 446 of SEQ ID NO: 11. In an embodiment, the peptide comprises the amino acid sequence of SEQ ID NO: 5 and the peptide is positioned immediately C-terminal to an amino acid in the parental capsid corresponding to amino acid 446 of SEQ ID NO: 11. In an embodiment, the peptide comprises the amino acid sequence of SEQ ID NO: 6 and the peptide is positioned immediately C-terminal to an amino acid in the parental capsid corresponding to amino acid 446 of SEQ ID NO: 11.

In an embodiment, the peptide is positioned immediately C-terminal to an amino acid in the parental capsid corresponding to amino acid 447 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 447 of SEQ ID NO: 11. In an embodiment, the peptide comprises the amino acid sequence of SEQ ID NO: 2 and the peptide is positioned immediately C-terminal to an amino acid in the parental capsid corresponding to amino acid 447 of SEQ ID NO: 11. In an embodiment, the peptide comprises the amino acid sequence of SEQ ID NO: 3 and the peptide is positioned immediately C-terminal to an amino acid in the parental capsid corresponding to amino acid 447 of SEQ ID NO: 11. In an embodiment, the peptide comprises the amino acid sequence of SEQ ID NO: 4 and the peptide is positioned immediately C-terminal to an amino acid in the parental capsid corresponding to amino acid 447 of SEQ ID NO: 11. In an embodiment, the peptide comprises the amino acid sequence of SEQ ID NO: 5 and the peptide is positioned immediately C-terminal to an amino acid in the parental capsid corresponding to amino acid 447 of SEQ ID NO: 11. In an embodiment, the peptide comprises the amino acid sequence of SEQ ID NO: 6 and the peptide is positioned immediately C-terminal to an amino acid in the parental capsid corresponding to amino acid 447 of SEQ ID NO: 11.

In an embodiment, the peptide is positioned immediately C-terminal to an amino acid in the parental capsid corresponding to amino acid 572 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 572 of SEQ ID NO: 11. In an embodiment, the peptide comprises the amino acid sequence of SEQ ID NO: 2 and the peptide is positioned immediately C-terminal to an amino acid in the parental capsid corresponding to amino acid 572 of SEQ ID NO: 11. In an embodiment, the peptide comprises the amino acid sequence of SEQ ID NO: 3 and the peptide is positioned immediately C-terminal to an amino acid in the parental capsid corresponding to amino acid 572 of SEQ ID NO: 11. In an embodiment, the peptide comprises the amino acid sequence of SEQ ID NO: 4 and the peptide is positioned immediately C-terminal to an amino acid in the parental capsid corresponding to amino acid 572 of SEQ ID NO: 11. In an embodiment, the peptide comprises the amino acid sequence of SEQ ID NO: 5 and the peptide is positioned immediately C-terminal to an amino acid in the parental capsid corresponding to amino acid 572 of SEQ ID NO: 11. In an embodiment, the peptide comprises the amino acid sequence of SEQ ID NO: 6 and the peptide is positioned immediately C-terminal to an amino acid in the parental capsid corresponding to amino acid 572 of SEQ ID NO: 11.

In an embodiment, the peptide is positioned immediately C-terminal to an amino acid in the parental capsid corresponding to amino acid 573 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 573 of SEQ ID NO: 11. In an embodiment, the peptide comprises the amino acid sequence of SEQ ID NO: 2 and the peptide is positioned immediately C-terminal to an amino acid in the parental capsid corresponding to amino acid 573 of SEQ ID NO: 11. In an embodiment, the peptide comprises the amino acid sequence of SEQ ID NO: 3 and the peptide is positioned immediately C-terminal to an amino acid in the parental capsid corresponding to amino acid 573 of SEQ ID NO: 11. In an embodiment, the peptide comprises the amino acid sequence of SEQ ID NO: 4 and the peptide is positioned immediately C-terminal to an amino acid in the parental capsid corresponding to amino acid 573 of SEQ ID NO: 11. In an embodiment, the peptide comprises the amino acid sequence of SEQ ID NO: 5 and the peptide is positioned immediately C-terminal to an amino acid in the parental capsid corresponding to amino acid 573 of SEQ ID NO: 11. In an embodiment, the peptide comprises the amino acid sequence of SEQ ID NO: 6 and the peptide is positioned immediately C-terminal to an amino acid in the parental capsid corresponding to amino acid 573 of SEQ ID NO: 11.

In an embodiment, the peptide is positioned immediately C-terminal to an amino acid in the parental capsid corresponding to amino acid 574 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 574 of SEQ ID NO: 11. In an embodiment, the peptide comprises the amino acid sequence of SEQ ID NO: 2 and the peptide is positioned immediately C-terminal to an amino acid in the parental capsid corresponding to amino acid 574 of SEQ ID NO: 11. In an embodiment, the peptide comprises the amino acid sequence of SEQ ID NO: 3 and the peptide is positioned immediately C-terminal to an amino acid in the parental capsid corresponding to amino acid 574 of SEQ ID NO: 11. In an embodiment, the peptide comprises the amino acid sequence of SEQ ID NO: 4 and the peptide is positioned immediately C-terminal to an amino acid in the parental capsid corresponding to amino acid 574 of SEQ ID NO: 11. In an embodiment, the peptide comprises the amino acid sequence of SEQ ID NO: 5 and the peptide is positioned immediately C-terminal to an amino acid in the parental capsid corresponding to amino acid 574 of SEQ ID NO: 11. In an embodiment, the peptide comprises the amino acid sequence of SEQ ID NO: 6 and the peptide is positioned immediately C-terminal to an amino acid in the parental capsid corresponding to amino acid 574 of SEQ ID NO: 11.

In an embodiment, the peptide is positioned immediately C-terminal to an amino acid in the parental capsid corresponding to amino acid 575 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 575 of SEQ ID NO: 11. In an embodiment, the peptide comprises the amino acid sequence of SEQ ID NO: 2 and the peptide is positioned immediately C-terminal to an amino acid in the parental capsid corresponding to amino acid 575 of SEQ ID NO: 11. In an embodiment, the peptide comprises the amino acid sequence of SEQ ID NO: 3 and the peptide is positioned immediately C-terminal to an amino acid in the parental capsid corresponding to amino acid 575 of SEQ ID NO: 11. In an embodiment, the peptide comprises the amino acid sequence of SEQ ID NO: 4 and the peptide is positioned immediately C-terminal to an amino acid in the parental capsid corresponding to amino acid 575 of SEQ ID NO: 11. In an embodiment, the peptide comprises the amino acid sequence of SEQ ID NO: 5 and the peptide is positioned immediately C-terminal to an amino acid in the parental capsid corresponding to amino acid 575 of SEQ ID NO: 11. In an embodiment, the peptide comprises the amino acid sequence of SEQ ID NO: 6 and the peptide is positioned immediately C-terminal to an amino acid in the parental capsid corresponding to amino acid 575 of SEQ ID NO: 11.

In an embodiment, the peptide is positioned immediately C-terminal to an amino acid in the parental capsid corresponding to amino acid 576 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 576 of SEQ ID NO: 11. In an embodiment, the peptide comprises the amino acid sequence of SEQ ID NO: 2 and the peptide is positioned immediately C-terminal to an amino acid in the parental capsid corresponding to amino acid 576 of SEQ ID NO: 11. In an embodiment, the peptide comprises the amino acid sequence of SEQ ID NO: 3 and the peptide is positioned immediately C-terminal to an amino acid in the parental capsid corresponding to amino acid 576 of SEQ ID NO: 11. In an embodiment, the peptide comprises the amino acid sequence of SEQ ID NO: 4 and the peptide is positioned immediately C-terminal to an amino acid in the parental capsid corresponding to amino acid 576 of SEQ ID NO: 11. In an embodiment, the peptide comprises the amino acid sequence of SEQ ID NO: 5 and the peptide is positioned immediately C-terminal to an amino acid in the parental capsid corresponding to amino acid 576 of SEQ ID NO: 11. In an embodiment, the peptide comprises the amino acid sequence of SEQ ID NO: 6 and the peptide is positioned immediately C-terminal to an amino acid in the parental capsid corresponding to amino acid 576 of SEQ ID NO: 11.

In an embodiment, the peptide is positioned immediately C-terminal to an amino acid in the parental capsid corresponding to amino acid 577 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 577 of SEQ ID NO: 11. In an embodiment, the peptide comprises the amino acid sequence of SEQ ID NO: 2 and the peptide is positioned immediately C-terminal to an amino acid in the parental capsid corresponding to amino acid 577 of SEQ ID NO: 11. In an embodiment, the peptide comprises the amino acid sequence of SEQ ID NO: 3 and the peptide is positioned immediately C-terminal to an amino acid in the parental capsid corresponding to amino acid 577 of SEQ ID NO: 11. In an embodiment, the peptide comprises the amino acid sequence of SEQ ID NO: 4 and the peptide is positioned immediately C-terminal to an amino acid in the parental capsid corresponding to amino acid 577 of SEQ ID NO: 11. In an embodiment, the peptide comprises the amino acid sequence of SEQ ID NO: 5 and the peptide is positioned immediately C-terminal to an amino acid in the parental capsid corresponding to amino acid 577 of SEQ ID NO: 11. In an embodiment, the peptide comprises the amino acid sequence of SEQ ID NO: 6 and the peptide is positioned immediately C-terminal to an amino acid in the parental capsid corresponding to amino acid 577 of SEQ ID NO: 11.

In an embodiment, the peptide is positioned immediately C-terminal to an amino acid in the parental capsid corresponding to amino acid 578 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 578 of SEQ ID NO: 11. In an embodiment, the peptide comprises the amino acid sequence of SEQ ID NO: 2 and the peptide is positioned immediately C-terminal to an amino acid in the parental capsid corresponding to amino acid 578 of SEQ ID NO: 11. In an embodiment, the peptide comprises the amino acid sequence of SEQ ID NO: 3 and the peptide is positioned immediately C-terminal to an amino acid in the parental capsid corresponding to amino acid 578 of SEQ ID NO: 11. In an embodiment, the peptide comprises the amino acid sequence of SEQ ID NO: 4 and the peptide is positioned immediately C-terminal to an amino acid in the parental capsid corresponding to amino acid 578 of SEQ ID NO: 11. In an embodiment, the peptide comprises the amino acid sequence of SEQ ID NO: 5 and the peptide is positioned immediately C-terminal to an amino acid in the parental capsid corresponding to amino acid 578 of SEQ ID NO: 11. In an embodiment, the peptide comprises the amino acid sequence of SEQ ID NO: 6 and the peptide is positioned immediately C-terminal to an amino acid in the parental capsid corresponding to amino acid 578 of SEQ ID NO: 11.

In an embodiment, the peptide is positioned immediately C-terminal to an amino acid in the parental capsid corresponding to amino acid 579 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 579 of SEQ ID NO: 11. In an embodiment, the peptide comprises the amino acid sequence of SEQ ID NO: 2 and the peptide is positioned immediately C-terminal to an amino acid in the parental capsid corresponding to amino acid 579 of SEQ ID NO: 11. In an embodiment, the peptide comprises the amino acid sequence of SEQ ID NO: 3 and the peptide is positioned immediately C-terminal to an amino acid in the parental capsid corresponding to amino acid 579 of SEQ ID NO: 11. In an embodiment, the peptide comprises the amino acid sequence of SEQ ID NO: 4 and the peptide is positioned immediately C-terminal to an amino acid in the parental capsid corresponding to amino acid 579 of SEQ ID NO: 11. In an embodiment, the peptide comprises the amino acid sequence of SEQ ID NO: 5 and the peptide is positioned immediately C-terminal to an amino acid in the parental capsid corresponding to amino acid 579 of SEQ ID NO: 11. In an embodiment, the peptide comprises the amino acid sequence of SEQ ID NO: 6 and the peptide is positioned immediately C-terminal to an amino acid in the parental capsid corresponding to amino acid 579 of SEQ ID NO: 11.

In an embodiment, the peptide is positioned immediately C-terminal to an amino acid in the parental capsid corresponding to amino acid 580 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 580 of SEQ ID NO: 11. In an embodiment, the peptide comprises the amino acid sequence of SEQ ID NO: 2 and the peptide is positioned immediately C-terminal to an amino acid in the parental capsid corresponding to amino acid 580 of SEQ ID NO: 11. In an embodiment, the peptide comprises the amino acid sequence of SEQ ID NO: 3 and the peptide is positioned immediately C-terminal to an amino acid in the parental capsid corresponding to amino acid 580 of SEQ ID NO: 11. In an embodiment, the peptide comprises the amino acid sequence of SEQ ID NO: 4 and the peptide is positioned immediately C-terminal to an amino acid in the parental capsid corresponding to amino acid 580 of SEQ ID NO: 11. In an embodiment, the peptide comprises the amino acid sequence of SEQ ID NO: 5 and the peptide is positioned immediately C-terminal to an amino acid in the parental capsid corresponding to amino acid 580 of SEQ ID NO: 11. In an embodiment, the peptide comprises the amino acid sequence of SEQ ID NO: 6 and the peptide is positioned immediately C-terminal to an amino acid in the parental capsid corresponding to amino acid 580 of SEQ ID NO: 11.

In an embodiment, the peptide is positioned immediately C-terminal to an amino acid in the parental capsid corresponding to amino acid 581 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 581 of SEQ ID NO: 11. In an embodiment, the peptide comprises the amino acid sequence of SEQ ID NO: 2 and the peptide is positioned immediately C-terminal to an amino acid in the parental capsid corresponding to amino acid 581 of SEQ ID NO: 11. In an embodiment, the peptide comprises the amino acid sequence of SEQ ID NO: 3 and the peptide is positioned immediately C-terminal to an amino acid in the parental capsid corresponding to amino acid 581 of SEQ ID NO: 11. In an embodiment, the peptide comprises the amino acid sequence of SEQ ID NO: 4 and the peptide is positioned immediately C-terminal to an amino acid in the parental capsid corresponding to amino acid 581 of SEQ ID NO: 11. In an embodiment, the peptide comprises the amino acid sequence of SEQ ID NO: 5 and the peptide is positioned immediately C-terminal to an amino acid in the parental capsid corresponding to amino acid 581 of SEQ ID NO: 11. In an embodiment, the peptide comprises the amino acid sequence of SEQ ID NO: 6 and the peptide is positioned immediately C-terminal to an amino acid in the parental capsid corresponding to amino acid 581 of SEQ ID NO: 11.

In an embodiment, the peptide is positioned immediately C-terminal to an amino acid in the parental capsid corresponding to amino acid 582 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 582 of SEQ ID NO: 11. In an embodiment, the peptide comprises the amino acid sequence of SEQ ID NO: 2 and the peptide is positioned immediately C-terminal to an amino acid in the parental capsid corresponding to amino acid 582 of SEQ ID NO: 11. In an embodiment, the peptide comprises the amino acid sequence of SEQ ID NO: 3 and the peptide is positioned immediately C-terminal to an amino acid in the parental capsid corresponding to amino acid 582 of SEQ ID NO: 11. In an embodiment, the peptide comprises the amino acid sequence of SEQ ID NO: 4 and the peptide is positioned immediately C-terminal to an amino acid in the parental capsid corresponding to amino acid 582 of SEQ ID NO: 11. In an embodiment, the peptide comprises the amino acid sequence of SEQ ID NO: 5 and the peptide is positioned immediately C-terminal to an amino acid in the parental capsid corresponding to amino acid 582 of SEQ ID NO: 11. In an embodiment, the peptide comprises the amino acid sequence of SEQ ID NO: 6 and the peptide is positioned immediately C-terminal to an amino acid in the parental capsid corresponding to amino acid 582 of SEQ ID NO: 11.

In an embodiment, the peptide is positioned immediately C-terminal to an amino acid in the parental capsid corresponding to amino acid 583 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 583 of SEQ ID NO: 11. In an embodiment, the peptide comprises the amino acid sequence of SEQ ID NO: 2 and the peptide is positioned immediately C-terminal to an amino acid in the parental capsid corresponding to amino acid 583 of SEQ ID NO: 11. In an embodiment, the peptide comprises the amino acid sequence of SEQ ID NO: 3 and the peptide is positioned immediately C-terminal to an amino acid in the parental capsid corresponding to amino acid 583 of SEQ ID NO: 11. In an embodiment, the peptide comprises the amino acid sequence of SEQ ID NO: 4 and the peptide is positioned immediately C-terminal to an amino acid in the parental capsid corresponding to amino acid 583 of SEQ ID NO: 11. In an embodiment, the peptide comprises the amino acid sequence of SEQ ID NO: 5 and the peptide is positioned immediately C-terminal to an amino acid in the parental capsid corresponding to amino acid 583 of SEQ ID NO: 11. In an embodiment, the peptide comprises the amino acid sequence of SEQ ID NO: 6 and the peptide is positioned immediately C-terminal to an amino acid in the parental capsid corresponding to amino acid 583 of SEQ ID NO: 11.

In an embodiment, the peptide is positioned immediately C-terminal to an amino acid in the parental capsid corresponding to amino acid 584 of SEQ ID NO: 11.

In an embodiment, the peptide comprises the amino acid sequence of SEQ ID NO: 1 and the peptide is positioned immediately C-terminal to an amino acid in the parental capsid corresponding to amino acid 584 of SEQ ID NO: 11. In an embodiment, the peptide comprises the amino acid sequence of SEQ ID NO: 2 and the peptide is positioned immediately C-terminal to an amino acid in the parental capsid corresponding to amino acid 584 of SEQ ID NO: 11. In an embodiment, the peptide comprises the amino acid sequence of SEQ ID NO: 3 and the peptide is positioned immediately C-terminal to an amino acid in the parental capsid corresponding to amino acid 584 of SEQ ID NO: 11. In an embodiment, the peptide comprises the amino acid sequence of SEQ ID NO: 4 and the peptide is positioned immediately C-terminal to an amino acid in the parental capsid corresponding to amino acid 584 of SEQ ID NO: 11. In an embodiment, the peptide comprises the amino acid sequence of SEQ ID NO: 5 and the peptide is positioned immediately C-terminal to an amino acid in the parental capsid corresponding to amino acid 584 of SEQ ID NO: 11. In an embodiment, the peptide comprises the amino acid sequence of SEQ ID NO: 6 and the peptide is positioned immediately C-terminal to an amino acid in the parental capsid corresponding to amino acid 584 of SEQ ID NO: 11.

In an embodiment, the recombinant AAV capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-737 of SEQ ID NO: 12, wherein amino acids 445-456 are GGHKAKGPRKLG (SEQ ID NO: 1); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-737 of SEQ ID NO: 13, wherein amino acids 578-589 are GGHKAKGPRKLG (SEQ ID NO: 1); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-734 of SEQ ID NO: 14, wherein amino acids 445-453 are GGHAIYPRH (SEQ ID NO: 2); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-734 of SEQ ID NO: 15, wherein amino acids 578-586 are GGHAIYPRH (SEQ ID NO: 2); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-732 of SEQ ID NO: 16, wherein amino acids 445-451 are RTIGPSV (SEQ ID NO: 3); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-732 of SEQ ID NO: 17, wherein amino acids 578-584 are RTIGPSV (SEQ ID NO: 3); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-732 of SEQ ID NO: 18, wherein amino acids 445-451 are LSSRLDA (SEQ ID NO: 4); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-732 of SEQ ID NO: 19, wherein amino acids 578-584 are LSSRLDA (SEQ ID NO: 4); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-737 of SEQ ID NO: 20, wherein amino acids 445-456 are THRPPMWSPVWP (SEQ ID NO: 5); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-737 of SEQ ID NO: 21, wherein amino acids 578-589 are THRPPMWSPVWP (SEQ ID NO: 5); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-737 of SEQ ID NO: 22, wherein amino acids 445-456 are FGQSSLPRDGPN (SEQ ID NO: 6); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 194-737 of SEQ ID NO: 23, wherein amino acids 578-589 are FGQSSLPRDGPN (SEQ ID NO: 6).

In an embodiment, the recombinant AAV capsid protein comprises an amino acid sequence that has at least 99% sequence identity to amino acids 194-737 of SEQ ID NO: 12, wherein amino acids 445-456 are GGHKAKGPRKLG (SEQ ID NO: 1); an amino acid sequence that has at least 99% sequence identity to amino acids 194-737 of SEQ ID NO: 13, wherein amino acids 578-578 are GGHKAKGPRKLG (SEQ ID NO: 1); an amino acid sequence that has at least 99% sequence identity to amino acids 194-734 of SEQ ID NO: 14, wherein amino acids 445-453 are GGHAIYPRH (SEQ ID NO: 2); an amino acid sequence that has at least 99% sequence identity to amino acids 194-734 of SEQ ID NO: 15, wherein amino acids 578-586 are GGHAIYPRH (SEQ ID NO: 2); an amino acid sequence that has at least 99% sequence identity to amino acids 194-732 of SEQ ID NO: 16, wherein amino acids 445-451 are RTIGPSV (SEQ ID NO: 3); an amino acid sequence that has at least 99% sequence identity to amino acids 194-732 of SEQ ID NO: 17, wherein amino acids 578-584 are RTIGPSV (SEQ ID NO: 3); an amino acid sequence that has at least 99% sequence identity to amino acids 194-732 of SEQ ID NO: 18, wherein amino acids 445-451 are LSSRLDA (SEQ ID NO: 4); an amino acid sequence that has at least 99% sequence identity to amino acids 194-732 of SEQ ID NO: 19, wherein amino acids 578-584 are LSSRLDA (SEQ ID NO: 4); an amino acid sequence that has at least 99% sequence identity to amino acids 194-737 of SEQ ID NO: 20, wherein amino acids 445-456 are THRPPMWSPVWP (SEQ ID NO: 5); an amino acid sequence that has at least 99% sequence identity to amino acids 194-737 of SEQ ID NO: 21, wherein amino acids 578-589 are THRPPMWSPVWP (SEQ ID NO: 5); an amino acid sequence that has at least 99% sequence identity to amino acids 194-737 of SEQ ID NO: 22, wherein amino acids 445-456 are FGQSSLPRDGPN (SEQ ID NO: 6); an amino acid sequence that has at least 99% sequence identity to amino acids 194-737 of SEQ ID NO: 23, wherein amino acids 578-589 are FGQSSLPRDGPN (SEQ ID NO: 6).

In an embodiment, the recombinant AAV capsid protein comprises an amino acid sequence that has 100% sequence identity to amino acids 194-737 of SEQ ID NO: 12; an amino acid sequence that has 100% sequence identity to amino acids 194-737 of SEQ ID NO: 13; an amino acid sequence that has 100% sequence identity to amino acids 194-734 of SEQ ID NO: 14; an amino acid sequence that has 100% sequence identity to amino acids 194-734 of SEQ ID NO: 15; an amino acid sequence that has 100% sequence identity to amino acids 194-732 of SEQ ID NO: 16; an amino acid sequence that has 100% sequence identity to amino acids 194-732 of SEQ ID NO: 17; an amino acid sequence that has 100% sequence identity to amino acids 194-732 of SEQ ID NO: 18; an amino acid sequence that has 100% sequence identity to amino acids 194-732 of SEQ ID NO: 19; an amino acid sequence that has 100% sequence identity to amino acids 194-737 of SEQ ID NO: 20; an amino acid sequence that has 100% sequence identity to amino acids 194-737 of SEQ ID NO: 21; an amino acid sequence that has 100% sequence identity to amino acids 194-737 of SEQ ID NO: 22; an amino acid sequence that has 100% sequence identity to amino acids 194-737 of SEQ ID NO: 23.

In an embodiment, the recombinant AAV capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-737 of SEQ ID NO: 12, wherein amino acids 445-456 are GGHKAKGPRKLG (SEQ ID NO: 1); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-737 of SEQ ID NO: 13, wherein amino acids 578-589 are GGHKAKGPRKLG (SEQ ID NO: 1); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-734 of SEQ ID NO: 14, wherein amino acids 445-453 are GGHAIYPRH (SEQ ID NO: 2); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-734 of SEQ ID NO: 15, wherein amino acids 578-586 are GGHAIYPRH (SEQ ID NO: 2); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-732 of SEQ ID NO: 16, wherein amino acids 445-451 are RTIGPSV (SEQ ID NO: 3); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-732 of SEQ ID NO: 17, wherein amino acids 578-584 are RTIGPSV (SEQ ID NO: 3); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-732 of SEQ ID NO: 18, wherein amino acids 445-451 are LSSRLDA (SEQ ID NO: 4); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-732 of SEQ ID NO: 19, wherein amino acids 578-584 are LSSRLDA (SEQ ID NO: 4); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-737 of SEQ ID NO: 20, wherein amino acids 445-456 are THRPPMWSPVWP (SEQ ID NO: 5); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-737 of SEQ ID NO: 21, wherein amino acids 578-589 are THRPPMWSPVWP (SEQ ID NO: 5); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-737 of SEQ ID NO: 22, wherein amino acids 445-456 are FGQSSLPRDGPN (SEQ ID NO: 6); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 138-737 of SEQ ID NO: 23, wherein amino acids 578-89 are FGQSSLPRDGPN (SEQ ID NO: 6).

In an embodiment, the recombinant AAV capsid protein comprises an amino acid sequence that has at least 99% sequence identity to amino acids 138-737 of SEQ ID NO: 12, wherein amino acids 445-456 are GGHKAKGPRKLG (SEQ ID NO: 1); an amino acid sequence that has at least 99% sequence identity to amino acids 138-737 of SEQ ID NO: 13, wherein amino acids 578-589 are GGHKAKGPRKLG (SEQ ID NO: 1); an amino acid sequence that has at least 99% sequence identity to amino acids 138-734 of SEQ ID NO: 14, wherein amino acids 445-453 are GGHAIYPRH (SEQ ID NO: 2); an amino acid sequence that has at least 99% sequence identity to amino acids 138-734 of SEQ ID NO: 15, wherein amino acids 578-586 are GGHAIYPRH (SEQ ID NO: 2); an amino acid sequence that has at least 99% sequence identity to amino acids 138-732 of SEQ ID NO: 16, wherein amino acids 445-451 are RTIGPSV (SEQ ID NO: 3); an amino acid sequence that has at least 99% sequence identity to amino acids 138-732 of SEQ ID NO: 17, wherein amino acids 578-584 are RTIGPSV (SEQ ID NO: 3); an amino acid sequence that has at least 99% sequence identity to amino acids 138-732 of SEQ ID NO: 18, wherein amino acids 445-451 are LSSRLDA (SEQ ID NO: 4); an amino acid sequence that has at least 99% sequence identity to amino acids 138-732 of SEQ ID NO: 19, wherein amino acids 578-584 are LSSRLDA (SEQ ID NO: 4); an amino acid sequence that has at least 99% sequence identity to amino acids 138-737 of SEQ ID NO: 20, wherein amino acids 445-456 are THRPPMWSPVWP (SEQ ID NO: 5); an amino acid sequence that has at least 99% sequence identity to amino acids 138-737 of SEQ ID NO: 21, wherein amino acids 578-589 are THRPPMWSPVWP (SEQ ID NO: 5); an amino acid sequence that has at least 99% sequence identity to amino acids 138-737 of SEQ ID NO: 22, wherein amino acids 445-456 are FGQSSLPRDGPN (SEQ ID NO: 6); an amino acid sequence that has at least 99% sequence identity to amino acids 138-737 of SEQ ID NO: 23, wherein amino acids 578-89 are FGQSSLPRDGPN (SEQ ID NO: 6).

In an embodiment, the recombinant AAV capsid protein comprises an amino acid sequence that has 100% sequence identity to amino acids 138-737 of SEQ ID NO: 12; an amino acid sequence that has 100% sequence identity to amino acids 138-737 of SEQ ID NO: 13; an amino acid sequence that has 100% sequence identity to amino acids 138-734 of SEQ ID NO: 14; an amino acid sequence that has 100% sequence identity to amino acids 138-734 of SEQ ID NO: 15; an amino acid sequence that has 100% sequence identity to amino acids 138-732 of SEQ ID NO: 16; an amino acid sequence that has 100% sequence identity to amino acids 138-732 of SEQ ID NO: 17; an amino acid sequence that has 100% sequence identity to amino acids 138-732 of SEQ ID NO: 18; an amino acid sequence that has 100% sequence identity to amino acids 138-732 of SEQ ID NO: 19; an amino acid sequence that has 100% sequence identity to amino acids 138-737 of SEQ ID NO: 20; an amino acid sequence that has 100% sequence identity to amino acids 138-737 of SEQ ID NO: 21; an amino acid sequence that has 100% sequence identity to amino acids 138-737 of SEQ ID NO: 22; an amino acid sequence that has 100% sequence identity to amino acids 138-737 of SEQ ID NO: 23.

In an embodiment, the recombinant AAV capsid protein comprises an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-737 of SEQ ID NO: 12, wherein amino acids 445-456 are GGHKAKGPRKLG (SEQ ID NO: 1); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-737 of SEQ ID NO: 13, wherein amino acids 578-589 are GGHKAKGPRKLG (SEQ ID NO: 1); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-734 of SEQ ID NO: 14, wherein amino acids 445-453 are GGHAIYPRH (SEQ ID NO: 2); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-734 of SEQ ID NO: 15, wherein amino acids 578-586 are GGHAIYPRH (SEQ ID NO: 2); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-732 of SEQ ID NO: 16, wherein amino acids 445-451 are RTIGPSV (SEQ ID NO: 3); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-732 of SEQ ID NO: 17, wherein amino acids 578-584 are RTIGPSV (SEQ ID NO: 3); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-732 of SEQ ID NO: 18, wherein amino acids 445-451 are LSSRLDA (SEQ ID NO: 4); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-732 of SEQ ID NO: 19, wherein amino acids 578-584 are LSSRLDA (SEQ ID NO: 4); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-737 of SEQ ID NO: 20, wherein amino acids 445-456 are THRPPMWSPVWP (SEQ ID NO: 5); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-737 of SEQ ID NO: 21, wherein amino acids 578-589 are THRPPMWSPVWP (SEQ ID NO: 5); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-737 of SEQ ID NO: 22, wherein amino acids 445-456 are FGQSSLPRDGPN (SEQ ID NO: 6); an amino acid sequence that has at least 80%, 81%, 82%, 83%, 84%, 85%, 86%, 87%, 88%, 89%, 90%, 91%, 92%, 93%, 94%, 95%, 96%, 97%, or 98% sequence identity to amino acids 1-737 of SEQ ID NO: 23, wherein amino acids 578-89 are FGQSSLPRDGPN (SEQ ID NO: 6).

In an embodiment, the recombinant AAV capsid protein comprises an amino acid sequence that has at least 99% sequence identity to amino acids 1-737 of SEQ ID NO: 12, wherein amino acids 445-456 are GGHKAKGPRKLG (SEQ ID NO: 1); an amino acid sequence that has at least 99% sequence identity to amino acids 1-737 of SEQ ID NO: 13, wherein amino acids 578-589 are GGHKAKGPRKLG (SEQ ID NO: 1); an amino acid sequence that has at least 99% sequence identity to amino acids 1-734 of SEQ ID NO: 14, wherein amino acids 445-453 are GGHAIYPRH (SEQ ID NO: 2); an amino acid sequence that has at least 99% sequence identity to amino acids 1-734 of SEQ ID NO: 15, wherein amino acids 578-586 are GGHAIYPRH (SEQ ID NO: 2); an amino acid sequence that has at least 99% sequence identity to amino acids 1-732 of SEQ ID NO: 16, wherein amino acids 445-451 are RTIGPSV (SEQ ID NO: 3); an amino acid sequence that has at least 99% sequence identity to amino acids 1-732 of SEQ ID NO: 17, wherein amino acids 578-584 are RTIGPSV (SEQ ID NO: 3); an amino acid sequence that has at least 99% sequence identity to amino acids 1-732 of SEQ ID NO: 18, wherein amino acids 445-451 are LSSRLDA (SEQ ID NO: 4); an amino acid sequence that has at least 99% sequence identity to amino acids 1-732 of SEQ ID NO: 19, wherein amino acids 578-584 are LSSRLDA (SEQ ID NO: 4); an amino acid sequence that has at least 99% sequence identity to amino acids 1-737 of SEQ ID NO: 20, wherein amino acids 445-456 are THRPPMWSPVWP (SEQ ID NO: 5); an amino acid sequence that has at least 99% sequence identity to amino acids 1-737 of SEQ ID NO: 21, wherein amino acids 578-589 are THRPPMWSPVWP (SEQ ID NO: 5); an amino acid sequence that has at least 99% sequence identity to amino acids 1-737 of SEQ ID NO: 22, wherein amino acids 445-456 are FGQSSLPRDGPN (SEQ ID NO: 6); an amino acid sequence that has at least 99% sequence identity to amino acids 1-737 of SEQ ID NO: 23, wherein amino acids 578-89 are FGQSSLPRDGPN (SEQ ID NO: 6).

In an embodiment, the recombinant AAV capsid protein comprises an amino acid sequence selected from the group consisting of SEQ ID NOs: 12-23. In an embodiment, the amino acid sequence of the recombinant AAV capsid protein consists of an amino acid sequence selected from the group consisting of SEQ ID NOs: 12-23.

In an embodiment, the peptide is linked (i.e., connected by a peptide bond) directly to the amino acids of the parental AAV capsid protein. In an embodiment, the peptide is linked to the amino acids of the parental AAV capsid protein via an amino acid linker. In an embodiment, the peptide comprises an N-terminal linker. In an embodiment, the peptide comprises a C-terminal linker. In an embodiment, the peptide comprises an N-terminal linker and a C-terminal linker. Suitable linkers include, without limitation, a G linker (AG), GS linker (GS), and a GG linker (GG), TG, GLS, or GQSG.

In an embodiment, the recombinant AAV capsid proteins disclosed herein bind to a protein that is expressed on the cell membrane of a muscle cell, microglia, astrocyte, neuron, blood brain barrier endothelial cell (BBB EC), pericyte, oligodendrocyte, ependymal cell, pyramidal neuron, rosehip neuron, purkinje cell, or cardiomyocyte. In an embodiment, the recombinant AAV capsid proteins disclosed herein bind to a protein selected from the group consisting of α-integrins or β-integrins (e.g., integrin alpha 7 (ITGA7)), αvβ1, αvβ3, αvβ5, αvβ6, αvβ8, α5β1, αIIbβ, α8β1) the transferrin receptor (Tflr), insulin receptor (INSR/CD220), ADP-ribosyltransferase 1 (ART1), and cadherin 15 (CDH15).

Amino acid sequences of exemplary recombinant AAV capsid proteins provided herein are set forth in Table 2 below.

TABLE 2
Amino acid sequences of exemplary recombinant AAV capsid proteins

| | SEQ ID | |
|---|---|---|
| Description | NO | Amino acid sequence |
| AAV5 (no peptide | 10 | MSFVDHPPDWLEEVGEGLREFLGLEAGPPKPKPNQQHQDQARGLV |
| insertion) | | LPGYNYLGPGNGLDRGEPVNRADEVAREHDISYNEQLEAGDNPYL |
| | | KYNHADAEFQEKLADDTSFGGNLGKAVFQAKKRVLEPFGLVEEGA |
| | | KTAPTGKRIDDHFPKRKKARTEEDSKPSTSSDAEAGPSGSQQLQIPA |
| | | QPASSLGADTMSAGGGGPLGDNNQGADGVGNASGDWHCDSTWM |
| | | GDRVVTKSTRTWVLPSYNNHQYREIKSGSVDGSNANAYFGYSTPW |
| | | GYFDFNRFHSHWSPRDWQRLINNYWGFRPRSLRVKIFNIQVKEVTV |
| | | QDSTTTIANNLTSTVQVFTDDDYQLPYVVGNGTEGCLPAFPPQVFT |
| | | LPQYGYATLNRDNTENPTERSSFFCLEYFPSKMLRTGNNFEFTYNFE |
| | | EVPFHSSFAPSQNLFKLANPLVDQYLYRFVSTNNTGGVQFNKNLAG |
| | | RYANTYKNWFPGPMGRTQGWNLGSGVNRASVSAFATTNRMELEG |
| | | ASYQVPPQPNGMTNNLQGSNTYALENTMIFNSQPANPGTTATYLE |
| | | GNMLITSESETQPVNRVAYNVGGQMATNNQSSTTAPATGTYNLQEI |
| | | VPGSVWMERDVYLQGPIWAKIPETGAHFHPSPAMGGFGLKHPPPM |
| | | MLIKNTPVPGNITSFSDVPVSSFITQYSTGQVTVEMEWELKKENSKR |
| | | WNPEIQYTNNYNDPQFVDFAPDSTGEYRTTRPIGTRYLTRPL |
| AAV5.2 (no peptide | 11 | MAADGYLPDWLEDTLSEGIRQWWKLKPGPPPPKPAERHKDDSRGL |
| insertion) | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTTAPATGTYN |
| | | LQEIVPGSVWMERDVYLQGPIWAKIPETGAHFHPSPAMGGFGLKHP |
| | | PPMMLIKNTPVPGNITSFSDVPVSSFITQYSTGQVTVEMEWELKKEN |
| | | SKRWNPEIQYTNNYNDPQFVDFAPDSTGEYRTTRPIGTRYLTRPL |
| VYPep2, loop IV | 12 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHKAKGPR |
| | | KLGTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep2, loop VIII | 13 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHKAKGPR |
| | | KLGTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep3, loop IV | 14 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHAIYPRHT |
| | | GGVQFNKNLAGRYANTYKNWFPGPMGRTQGWNLGSGVNRASVS |
| | | AFATTNRMELEGASYQVPPQPNGMTNNLQGSNTYALENTMIFNSQ |
| | | PANPGTTATYLEGNMLITSESETQPVNRVAYNVGGQMATNNQSST |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep3, loop VIII | 15 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHAIYPRHT |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep4, loop IV | 16 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNRTIGPSVTGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep4, loop VIII | 17 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSRTIGPSVTTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop IV | 18 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNLSSRLDATGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop VIII | 19 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSLSSRLDATTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep6, loop IV | 20 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTHRPPMWSP |
| | | VWPTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep6, loop VIII | 21 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTHRPPMWSPV |
| | | WPTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop IV | 22 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNFGQSSLPRDG |
| | | PNTGGVQFNKNLAGRYANTYKNWFPGPMGRTQGWNLGSGVNRA |
| | | SVSAFATTNRMELEGASYQVPPQPNGMTNNLQGSNTYALENTMIF |
| | | NSQPANPGTTATYLEGNMLITSESETQPVNRVAYNVGGQMATNNQ |
| | | SSTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop VIII | 23 | MAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSFGQSSLPRDGP |
| | | NGGTTAPATGTYNLQEIVPGSVWMERDVYLQGPIWAKIPETGAHF |
| | | HPSPAMGGFGLKHPPPMMLIKNTPVPGNITSFSDVPVSSFITQYSTG |
| | | QVTVEMEWELKKENSKRWNPEIQYTNNYNDPQFVDFAPDSTGEYR |
| | | TTRPIGTRYLTRPL |

Additional embodiments of the recombinant AAV capsid proteins disclosed herein are listed in Table 3 below.

TABLE 3
Amino acid sequences of additional exemplary recombinant AAV capsid proteins

| | SEQ ID | |
|---|---|---|
| Description | NO | Amino acid sequence |
| VYPep2, loop IV | 7 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHKAKGPR |
| | | KLGTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep2, loop VIII | 8 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHKAKGPR |
| | | KLGTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep3, loop IV | 9 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNGGHAIYPRHT |
| | | GGVQFNKNLAGRYANTYKNWFPGPMGRTQGWNLGSGVNRASVS |
| | | AFATTNRMELEGASYQVPPQPNGMTNNLQGSNTYALENTMIFNSQ |
| | | PANPGTTATYLEGNMLITSESETQPVNRVAYNVGGQMATNNQSST |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep3, loop VIII | 24 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSGGHAIYPRHT |
| | | TAPATGTYNLQEIVPGSVWMERDVYLQGPIWAKIPETGAHFHPSPA |
| | | MGGFGLKHPPPMMLIKNTPVPGNITSFSDVPVSSFITQYSTGQVTVE |
| | | MEWELKKENSKRWNPEIQYTNNYNDPQFVDFAPDSTGEYRTTRPI |
| | | GTRYLTRPL |
| VYPep4, loop IV | 25 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNRTIGPSVTGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep4, loop VIII | 26 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSRTIGPSVTTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop IV | 27 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNLSSRLDATGG |
| | | VQFNKNLAGRYANTYKNWFPGPMGRTQGWNLGSGVNRASVSAF |
| | | ATTNRMELEGASYQVPPQPNGMTNNLQGSNTYALENTMIFNSQPA |
| | | NPGTTATYLEGNMLITSESETQPVNRVAYNVGGQMATNNQSSTTA |
| | | PATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep5, loop VIII | 28 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSLSSRLDATTAP |
| | | ATGTYNLQEIVPGSVWMERDVYLQGPIWAKIPETGAHFHPSPAMG |
| | | GFGLKHPPPMMLIKNTPVPGNITSFSDVPVSSFITQYSTGQVTVEME |
| | | WELKKENSKRWNPEIQYTNNYNDPQFVDFAPDSTGEYRTTRPIGTR |
| | | YLTRPL |
| VYPep6, loop IV | 29 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTHRPPMWSP |
| | | VWPTGGVQFNKNLAGRYANTYKNWFPGPMGRTQGWNLGSGVNR |
| | | ASVSAFATTNRMELEGASYQVPPQPNGMTNNLQGSNTYALENTMI |
| | | FNSQPANPGTTATYLEGNMLITSESETQPVNRVAYNVGGQMATNN |
| | | QSSTTAPATGTYNLQEIVPGSVWMERDVYLQGPIWAKIPETGAHFH |
| | | PSPAMGGFGLKHPPPMMLIKNTPVPGNITSFSDVPVSSFITQYSTGQ |
| | | VTVEMEWELKKENSKRWNPEIQYTNNYNDPQFVDFAPDSTGEYRT |
| | | TRPIGTRYLTRPL |
| VYPep6, loop VIII | 30 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSTHRPPMWSPV |
| | | WPTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop IV | 31 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNFGQSSLPRDG |
| | | PNTGGVQFNKNLAGRYANTYKNWFPGPMGRTQGWNLGSGVNRA |
| | | SVSAFATTNRMELEGASYQVPPQPNGMTNNLQGSNTYALENTMIF |
| | | NSQPANPGTTATYLEGNMLITSESETQPVNRVAYNVGGQMATNNQ |
| | | SSTTAPATGTYNLQEIVPGSVWMERDVYLQGPIWAKIPETGAHFHP |
| | | SPAMGGFGLKHPPPMMLIKNTPVPGNITSFSDVPVSSFITQYSTGQV |
| | | TVEMEWELKKENSKRWNPEIQYTNNYNDPQFVDFAPDSTGEYRTT |
| | | RPIGTRYLTRPL |
| VYPep11, loop VIII | 32 | TAADGYLPDWLEDTLSEGIRQWWKLKPGPPPPKPAERHKDDSRGL |
| insertion | | VLPGYKYLGPFNGLDKGEPVNEADAAALEHDKAYDRQLDSGDNP |
| | | YLKYNHADAEFQERLKEDTSFGGNLGRAVFQAKKRVLEPLGLVEE |
| | | PVKTAPTGKRIDDHFPKRKKARTEEDSKPSTSSDAEAGPSGSQQLQI |
| | | PAQPASSLGADTMSAGGGGPLGDNNQGADGVGNASGDWHCDST |
| | | WMGDRVVTKSTRTWVLPSYNNHQYREIKSGSVDGSNANAYFGYS |
| | | TPWGYFDFNRFHSHWSPRDWQRLINNYWGFRPRSLRVKIFNIQVKE |
| | | VTVQDSTTTIANNLTSTVQVFTDDDYQLPYVVGNGTEGCLPAFPPQ |
| | | VFTLPQYGYATLNRDNTENPTERSSFFCLEYFPSKMLRTGNNFEFTY |
| | | NFEEVPFHSSFAPSQNLFKLANPLVDQYLYRFVSTNNTGGVQFNKN |
| | | LAGRYANTYKNWFPGPMGRTQGWNLGSGVNRASVSAFATTNRME |
| | | LEGASYQVPPQPNGMTNNLQGSNTYALENTMIFNSQPANPGTTAT |
| | | YLEGNMLITSESETQPVNRVAYNVGGQMATNNQSSFGQSSLPRDGP |
| | | NGGTTAPATGTYNLQEIVPGSVWMERDVYLQGPIWAKIPETGAHF |
| | | HPSPAMGGFGLKHPPPMMLIKNTPVPGNITSFSDVPVSSFITQYSTG |
| | | QVTVEMEWELKKENSKRWNPEIQYTNNYNDPQFVDFAPDSTGEYR |
| | | TTRPIGTRYLTRPL |

### Polynucleotides, Vectors, and Methods of Producing AAV Capsid Proteins

In an aspect, provided herein is an isolated polynucleotide encoding a recombinant AAV capsid protein disclosed herein.

In an embodiment, the polynucleotide is optimized, e.g., by codon/RNA optimization, replacement with heterologous signal sequences, and/or elimination of mRNA instability elements. Methods to generate optimized polynucleotides for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid. Such methods can increase expression of the encoded capsid protein relative to the expression of the capsid encoded by polynucleotides that have not been optimized.

In an aspect, provided herein is a vector comprising a polynucleotide disclosed herein. Suitable vectors, include, without limitation, plasmids, viruses, cosmids, artificial chromosomes, linear DNA, and mRNA. In an embodiment, the vector is a plasmid or a viral vector. In an embodiment, the vector is a retrovirus vector, a herpes virus vector, a baculovirus vector, or an adenovirus vector. In an embodiment, the vector is an expression vector.

Vectors (e.g., expression vectors) can be introduced into cells (using any techniques known in the art) for propagation of the vector and/or for expression of an AAV capsid protein encoded by the vector. Accordingly, in another aspect, the instant disclosure provides a recombinant cell comprising a polynucleotide or a vector (e.g., an expression vector) disclosed herein. And further, in another aspect, the instant disclosure provides a method of producing an AAV capsid protein, the method comprising culturing the recombinant cell under conditions whereby the polynucleotide is expressed, and the capsid is produced.

A variety of host cells and expression vector systems can be utilized to express the capsid proteins described herein. In these expression systems, the coding sequences of interest can be produced and subsequently purified. These expression systems also represent cells which can, when transformed or transfected with the appropriate nucleotide coding sequences, express a capsid protein described herein in situ. These include but are not limited to microorganisms such as bacteria (e.g., E. coli and B. subtilis) transformed with, e.g., recombinant bacteriophage DNA, plasmid DNA, or cosmid DNA expression vectors containing capsid protein coding sequences; yeast (e.g., Saccharomyces pichia) transformed with, e.g., recombinant yeast expression vectors containing capsid protein coding sequences; insect cell systems infected with, e.g., recombinant virus expression vectors (e.g., baculovirus) containing capsid protein coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing capsid protein coding sequences; or mammalian cell systems (e.g., COS (e.g., COS1 or COS), CHO, BHK, MDCK, HEK 293, NS0, PER.C6, VERO, CRL7O3O, HsS78Bst, HeLa, NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In an embodiment, cells for expressing the capsid proteins described herein are human cells, e.g., human cell lines. In an embodiment, a mammalian expression vector is pOptiVEC™ or pcDNA3.3. In an embodiment, bacterial cells such as Escherichia coli, or eukaryotic cells (e.g., mammalian cells), are used for the expression of a capsid protein. For example, mammalian cells such as CHO or HEK293 cells, in conjunction with a vector such as the major intermediate early gene promoter element from human cytomegalovirus, is an effective expression system for capsid proteins disclosed herein. In an embodiment, insect cells (e.g., Sf9 cells) are used for the expression of a capsid protein.

In an insect system, Autographa californica nuclear polyhedrosis virus (AcNPV), for example, can be used as a vector to express foreign genes. The virus grows in Spodoptera frugiperda cells. The capsid protein coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the capsid protein coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region El or E3) will result in a recombinant virus that is viable and capable of expressing the capsid protein molecule in infected hosts (see, e.g., Logan J & Shenk T (1984) PNAS 81(12): 3655-9, which is herein incorporated by reference in its entirety). Specific initiation signals can also be required for efficient translation of inserted capsid protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544, which is herein incorporated by reference in its entirety). Such mammalian host cells include but are not limited to CHO, VERO, BHK, Hela, MDCK, HEK 293, NIH 3T3, W138, BT483, Hs578T, HTB2, BT2O, T-47D, NS0, CRL7O3O, COS (e.g., COS1 or COS), PER.C6, VERO, HsS78Bst, HEK-293T, HepG2, SP210, R1.1, B-W, L-M, BSC1, BSC40, YB/20, BMT10, and HsS78Bst cells.

For long-term, high-yield production of recombinant proteins, stable expression cells can be generated. For example, cell lines which stably express a capsid protein described herein can be engineered.

In an embodiment, rather than using expression vectors which contain viral origins of replication, host cells can be transformed with a polynucleotide (e.g., DNA or RNA) controlled by appropriate transcriptional regulatory elements (e.g., promoter, enhancer, sequences, transcription terminators, polyadenylation sites, etc.), and a selectable marker. Following the introduction of polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express a capsid protein described herein or a fragment thereof.

A number of selection systems can be used, including but not limited to the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034), and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; Morgan R A & Anderson W F (1993) Ann Rev Biochem 62: 191-217; and Nabel G J & Felgner P L (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150: 1-14, all of which are herein incorporated by reference in their entireties.

### AAV Compositions

In one aspect, provided herein is an rAAV composition comprising a capsid comprising a recombinant AAV capsid protein disclosed herein.

In an aspect, provided herein is an rAAV comprising a capsid comprising one or more recombinant capsid protein disclosed herein; and an rAAV genome.

In an embodiment, the rAAV genome comprises a transgene. In an embodiment, the transgene encodes a polypeptide. In an embodiment, the transgene encodes an antibody or a fragment thereof. In an embodiment, the transgene encodes an scFv.

The rAAVs disclosed herein generally comprise a recombinant genome (e.g., an rAAV genome) packaged within the capsid. The rAAV genome can be of any type that is capable of being packaged within an AAV capsid disclosed herein. For example, in an embodiment, the rAAV genome is a single-stranded DNA genome. In an embodiment, the rAAV genome is a self-complementary genome, for example as described in U.S. Pat. No. 7,790,154, which is hereby incorporated by reference in its entirety.

In an embodiment, the rAAV genome comprises a transgene. In an embodiment, the transgene encodes a therapeutic protein. In an embodiment, the transgene encodes an antibody or a fragment thereof (e.g., a Fab, scFv, or full-length antibody). In an embodiment, the transgene encodes an scFv, nanobody, or VHH. In an embodiment, the transgene encodes a non-coding RNA.

In an embodiment, the transgene encodes reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In an embodiment, the rAAV genome comprises a TRE operably linked to the transgene, to control expression of an RNA or polypeptide encoded by the transgene. In an embodiment, the TRE comprises a constitutive promoter. In an embodiment, the TRE is active in any mammalian cell (e.g., any human cell). In an embodiment, the TRE is active in a broad range of human cells. Such TREs may comprise constitutive promoter and/or enhancer elements, including any of those described herein, and any of those known to one of skill in the art. In an embodiment, the TRE comprises an inducible promoter. In an embodiment, the TRE is a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. Tissue-specific promoter and/or enhancer elements can be isolated from genes specifically expressed in the tissue by methods well known in the art.

Suitable promoters include, e.g., cytomegalovirus promoter (CMV) (Stinski et al., (1985) Journal of Virology 55(2): 431-441), CMV early enhancer/chicken 3-actin (CBA) promoter/rabbit β-globin intron (CAG) (Miyazaki et al., (1989) Gene 79(2): 269-277), hybrid form of the CBA promoter (CBh) (Gray et al., (2011) Hum Gene Ther. 22(9):1143-53), CB^{SB} (Jacobson et al., (2006) Molecular Therapy 13(6): 1074-1084), human elongation factor 1α promoter (EF1α) (Kim et al., (1990) Gene 91(2): 217-223), human phosphoglycerate kinase promoter (PGK) (Singer-Sam et al., (1984) Gene 32(3): 409-417), mitochondrial heavy-strand promoter (Lodeiro et al., (2012) PNAS 109(17): 6513-6518), ubiquitin promoter (Wulff et al., (1990) FEBS Letters 261: 101-105).

In an aspect, provided herein is a pharmaceutical composition comprising an rAAV disclosed herein together with a pharmaceutically acceptable excipient, adjuvant, diluent, vehicle or carrier, or a combination thereof. A “pharmaceutically acceptable carrier” includes any material which, when combined with an active ingredient of a composition, allows the ingredient to retain biological activity and without causing disruptive physiological reactions, such as an unintended immune reaction. Pharmaceutically acceptable carriers include water, phosphate buffered saline, emulsions such as oil/water emulsion, and wetting agents. Compositions comprising such carriers are formulated by well-known conventional methods such as those set forth in Remington's Pharmaceutical Sciences, current Ed., Mack Publishing Co., Easton Pa. 18042, USA; A. Gennaro (2000) “Remington: The Science and Practice of Pharmacy,” 20th Ed., Lippincott, Williams, & Wilkins; Pharmaceutical Dosage Forms and Drug Delivery Systems (1999) H. C. Ansel et al., 7th Ed., Lippincott, Williams, & Wilkins; and Handbook of Pharmaceutical Excipients (2000) A. H. Kibbe et al., 3rd eE. Amer. Pharmaceutical Assoc.

### Methods of Use

In an aspect, provided herein is a method of transducing a cell, the method comprising contacting the cell with an rAAV disclosed herein under conditions where the cell is transduced, and the transgene is expressed. In an embodiment, the cell is a muscle cell, microglia, astrocyte, neuron, blood brain barrier endothelial cell (BBB EC), pericyte, oligodendrocyte, ependymal cell, or cardiomyocyte. In an embodiment, the neuron is a pyramidal neuron, rosehip neuron, or purkinje cell.

In an embodiment, provided herein is a method for expressing a transgene in a cell, the method generally comprising contacting the cell with such an rAAV under conditions whereby the cell is transduced, and the transgene is expressed. The transgene can encode a polypeptide, such as an antibody or a fragment thereof (e.g., an scFv), as described herein. Accordingly, in an embodiment, provided herein is a method for producing a polypeptide, such as an antibody or a fragment thereof (e.g., an scFv) in a cell, the method generally comprising contacting the cell with such an rAAV under conditions whereby the cell is transduced and the polypeptide, such as an antibody or a fragment thereof (e.g., an scFv), is produced.

In an aspect, provided herein is a method of delivering a transgene to a cell, the method comprising contacting the cell with an rAAV disclosed herein, under conditions where the cell is transduced, and the transgene is expressed. The rAAV disclosed herein can be used to transduce cells in vitro, in vivo and ex vivo. Cells suitable for being transduced by an rAAV disclosed herein include, without limitation, blood, liver, heart, joint tissue, muscle, brain, spinal cord, kidney, or lung cells. In an embodiment, the cell is a muscle cell, microglia, astrocyte, neuron, blood brain barrier endothelial cell (BBB EC), pericyte, oligodendrocyte, ependymal cell, or cardiomyocyte. In an embodiment, the neuron is a pyramidal neuron, rosehip neuron, or purkinje cell. In an embodiment, an rAAV provided herein is used to transduce cells in vivo in a subject who has a disease associated with muscle, brain, or spinal cord tissue.

In an aspect, provided herein is a method of delivering a transgene to muscle, brain or spinal cord tissue in a subject in need thereof, the method comprising administering an rAAV disclosed to the subject.

In an aspect, provided herein is a method of treating a neuromuscular or neurodegenerative disease or disorder in a subject in need thereof, comprising administering an effective amount of an rAAV disclosed herein to the subject. In an embodiment, the neuromuscular disease is selected from the group consisting of myopathy, hereditary cardiomyopathy, metabolic myopathy, distal myopathy, muscular dystrophy, congenital myopathy, spinal muscular atrophy (SMA), motor neuron disease, congenital myopathy, congenital muscular dystrophy, motor neuron disease, Duchenne muscular dystrophy, Becker muscular dystrophy, limb-girdle muscular dystrophies, myotonic dystrophy, myotubular myopathy, centronuclear myopathy, nemaline myopathy, selenoprotein N-related myopathy, Pompe disease, glycogen storage disease III, spinal muscular atrophy, amyotrophic lateral sclerosis (ALS), Charcot-Marie-Tooth disease, multiple sclerosis, myositis, polymyositis, and dermatomyositis.

In an embodiment, the neurodegenerative disease or disorder is selected from the group consisting of motor neuron disease (MND), parkinsonism syndrome, Alzheimer's dementia, progressive supranuclear palsy (PSP), Huntington's disease, multiple system atrophy (MSA), spinocerebellar ataxia (SCA), and Friedreich's ataxia.

In an embodiment, the rAAV disclosed herein can be administered to a subject (e.g., a human subject) by all routes suitable for an rAAV, including, without limitation, intravenously, intraperitoneally, subcutaneously, intramuscularly, intrathecally, or intradermally.

In an aspect, provided herein is an rAAV as disclosed herein for use in medicine. In an aspect, provided herein is an rAAV as disclosed herein for use as therapy. In an aspect, provided herein is an rAAV as disclosed herein for use as a medicament.

### Adeno-Associated Virus Packaging Systems

In an aspect, provided herein is a packaging system for recombinant preparation of a recombinant adeno-associated virus (rAAV) disclosed herein. Such packaging systems generally comprise: a first nucleotide sequence encoding one or more AAV Rep proteins; a second nucleotide sequence encoding a capsid protein of any recombinant AAV capsid disclosed herein; and a third nucleotide sequence comprising any of AAV genome sequence disclosed herein, wherein the packaging system is operative in a cell for enclosing the AAV genome in the capsid to form the rAAV.

In an embodiment, the packaging system comprises a first vector comprising the first nucleotide sequence encoding the one or more AAV Rep proteins and the second nucleotide sequence encoding the AAV capsid protein, and a second vector comprising the third nucleotide sequence comprising the rAAV genome. As used in the context of a packaging system as described herein, a “vector” refers to a nucleic acid molecule that is a vehicle for introducing nucleic acids into a cell (e.g., a plasmid, a virus, a cosmid, an artificial chromosome, etc.).

Any AAV Rep protein can be employed in the packaging systems disclosed herein. In an embodiment of the packaging system, the Rep nucleotide sequence encodes an AAV2 Rep protein. Suitable AAV2 Rep proteins may include, without limitation, Rep 78/68 or Rep 68/52.

In an embodiment, the packaging system further comprises a fourth nucleotide sequence comprising one or more helper virus genes. In an embodiment, the fourth nucleotide sequence comprises adenoviral E2, E4, and VA genes. In an embodiment, the packaging system further comprises a third vector (e.g., a helper virus vector), comprising the fourth nucleotide sequence. The third vector may be an independent third vector, integral with the first vector, or integral with the second vector.

In an embodiment of the packaging system, the helper virus is selected from the group consisting of adenovirus, herpes virus (including herpes simplex virus (HSV)), poxvirus (such as vaccinia virus), cytomegalovirus (CMV), and baculovirus. In an embodiment where the helper virus is adenovirus, the adenovirus genome comprises one or more adenovirus RNA genes selected from the group consisting of E1, E2, E4, and VA. In an embodiment, the adenovirus genome comprises one or more adenovirus RNA genes selected from the group consisting of E2, E4, and VA. In an embodiment where the helper virus is HSV, the HSV genome comprises one or more HSV genes selected from the group consisting of UL5/8/52, ICPO, ICP4, ICP22, and UL30/UL42.

In an embodiment of the packaging system, the first, second, and/or third vector are contained within one or more plasmids. In an embodiment, the first vector and the third vector are contained within a first plasmid. In an embodiment, the second vector and the third vector are contained within a second plasmid.

In an embodiment of the packaging system, the first, second, and/or third vector are contained within one or more recombinant helper viruses. In an embodiment, the first vector and the third vector are contained within a recombinant helper virus. In an embodiment, the second vector and the third vector are contained within a recombinant helper virus.

In an aspect, provided herein is a method for recombinant preparation of an rAAV as described herein, wherein the method comprises transfecting or transducing a cell with a packaging system as described herein under conditions operative for enclosing the rAAV genome in the capsid to form the rAAV as described herein. Exemplary methods for recombinant preparation of an rAAV include transient transfection (e.g., with one or more transfection plasmids containing a first, and a second, and optionally a third vector as described herein), viral infection (e.g., with one or more recombinant helper viruses, such as adenovirus, poxvirus (such as vaccinia virus), herpes virus (including HSV, cytomegalovirus, or baculovirus), containing a first, and a second, and optionally a third vector as described herein), and stable producer cell line transfection or infection (e.g., with a stable producer cell, such as a mammalian or insect cell, containing a Rep nucleotide sequence encoding one or more AAV Rep proteins and/or a Cap nucleotide sequence encoding one or more capsid proteins as described herein, and with a transfer genome as described herein being delivered in the form of a plasmid or a recombinant helper virus).

Accordingly, the instant disclosure provides a packaging system for preparation of an rAAV, wherein the packaging system comprises: a first nucleotide sequence encoding one or more AAV Rep proteins; a second nucleotide sequence encoding an AAV capsid protein disclosed herein; a third nucleotide sequence comprising an rAAV genome sequence described herein; and optionally a fourth nucleotide sequence comprising one or more helper virus genes (e.g., adenoviral E2, E4, and VA genes).

### EXAMPLES

### Example 1: Production of the Modified Capsids

Recombinant AAV capsids that efficiently cross the BBB were developed by inserting a peptide within the amino acid sequence of a parental AAV capsid protein.

a. AAV5.2 Capsid

A chimeric AAV capsid (AAV5.2, SEQ ID NO: 11) was used as the parental capsid protein to generate recombinant AAV capsid proteins. An AAV capsid is encoded by three viral proteins (VPs), VP1, VP2, and VP3, which are incorporated into a 60-subunit capsid in a 1:1:10 ratio. The AAV5.2 capsid is an engineered variant of AAV2 and AAV5 capsids, which includes a VP1 sequence from AAV2 and a VP2/3 sequence from AAV5.

b. Design of Capsids

The VP1 and VP2 unique sequences are located on the inside of the capsid and contain a phospholipase (PLA2) domain (in VP1) and nuclear localization signal (VP1 and VP2). The VP3 sequence (which is contained in all three VPs) is the primary protein sequence which constitutes the surface topology of the AAV capsid, which in turn dictates tropism and antigenicity. The VP3 topology contains a core eight-stranded (βB to βI)-barrel motif and large loop insertions between the β-strands which form the surface of the capsid. Conformational variations in these nine surface loops (VR-I to VR-IX) define the unique surface features for the distinct AAV serotypes. Accordingly, the peptide insertions described here are inserted in these externally facing loops. Specifically, loops IV and VIII were selected for peptide insertion sites.

Position 443, located at the apex of loop IV on the AAV5 (SEQ ID NO: 10) or position 444 on AAV5.2 (SEQ ID NO: 11) capsid, was selected as an insertion site in loop IV, the peptides were inserted immediately C-terminal to these amino acid positions. Position 576, located at the apex of loop VIII on the AAV5 (SEQ ID NO: 10) or position 577 on AAV5.2 (SEQ ID NO 11) capsid, was selected as the insertion site in loop VIII, the peptides were inserted immediately C-terminal to these amino acid positions. Insertion of a peptide immediately C-terminal to these positions should have minimal effect on capsid integrity because the remaining VP residue interactions remain intact at the base of the capsid.

Six different peptides (SEQ ID NOs: 1-6; see Table 1) were used to engineer the recombinant AAV capsid proteins. Specifically, polynucleotides encoding capsid proteins comprising a parental AAV5.2 capsid protein with a peptide insertion from Table 1 were synthesized and inserted into a plasmid vector. Each AAV capsid protein was engineered to contain only one peptide insertion, either immediately C-terminal to position 444 in loop IV or 577 in loop VIII according to AAV5.2 (SEQ ID NO: 11) numbering. The amino acid sequences of the resulting recombinant AAV capsid proteins (SEQ ID NOs: 12-23) are disclosed in Table 2.

### Example 2: Transduction of Human Myotubes

The transduction efficiency of the recombinant AAV capsids was further analyzed by transducing human myotubes with the capsids.

Human skeletal muscle cells (CC-2580, Lonza) were plated at a cell density of 50,000 cells per well of a 24 well plate. Differentiation of myoblasts was induced when the cells reached 85-90% confluency by replacing myoblast proliferation media with myoblast (low serum) differentiation media. Myotube differentiation was carried out for 7 days with differentiation medium being partially replaced every 2 days. AAV transduction of the myotubes was performed at an MOI of 1×10⁶ per cell, mixed with 50 uL of PBS at the start of differentiation and negative controls were treated with 50 uL of PBS only. One day post-transduction, medium was refreshed. For analysis of transduction efficacy, GFP+ cells were monitored overtime with the EVOS FLoid Imaging System (Cat. No. 447116). One-week post-transduction, the transduced myotubes were imaged using the EVOS FLoid Imaging System (Cat. No. 447116) at 20× magnification and later harvested and frozen for further quantitative analyses.

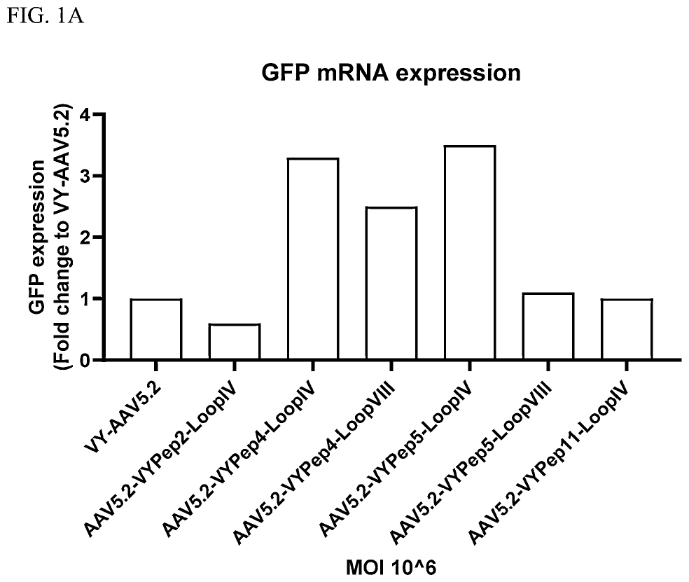

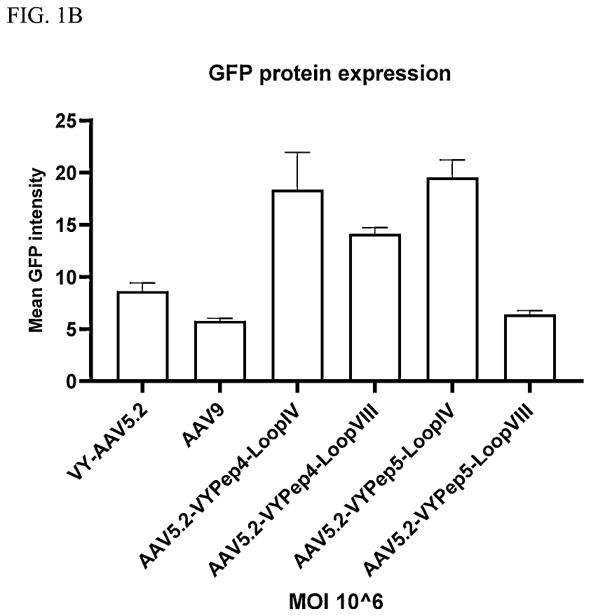

The results shown in FIG. 1A demonstrate that several of the recombinant AAV capsids show successful transduction of human myotubes and that some of the capsids show increased transduction of myotubes when the peptide is inserted in loop IV compared to loop VIII (VYPep4 and VYPep5). Further, the results shown in FIG. 1B demonstrate that recombinant AAV capsids comprising VYPep4 or VYPep5 at position 444 in loop IV or 577 in loop VIII successfully transduced myotubes.

### Example 3: Transduction of Human Astrocytes and Motor Neurons

The transduction efficiency of the recombinant AAV capsids was further analyzed by transducing human astrocytes and motor neurons with the capsids. The AAV5.2 capsid comprising VYPep4, VYPep5, or VYPep11 in either loop IV or loop VIII were analyzed in the following assays.

Human astrocytes and motor neurons were plated at a cell density of 50,000 cells per well of a 24 well plate. AAV transduction of the astrocytes and motor neurons was performed at an MOI of 1×10⁴ or 1×10⁶ per cell, mixed with 50 uL of PBS and negative controls were treated with 50 uL of PBS only. One day post-transduction, medium was refreshed. For analysis of transduction efficacy, GFP+ cells were monitored overtime with the EVOS FLoid Imaging System (Cat. No. 447116). One-week post-transduction, the transduced astrocytes and motor neurons were imaged using the EVOS FLoid Imaging System (Cat. No. 447116) at 20× magnification and later harvested and frozen for further quantitative analyses.

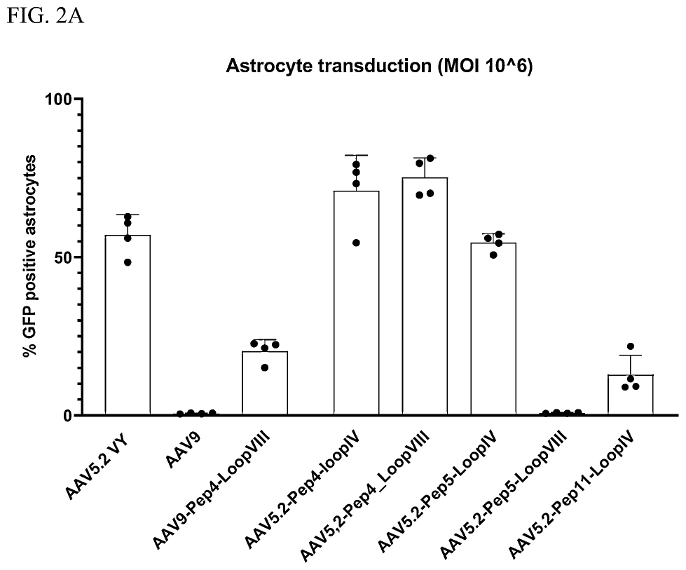

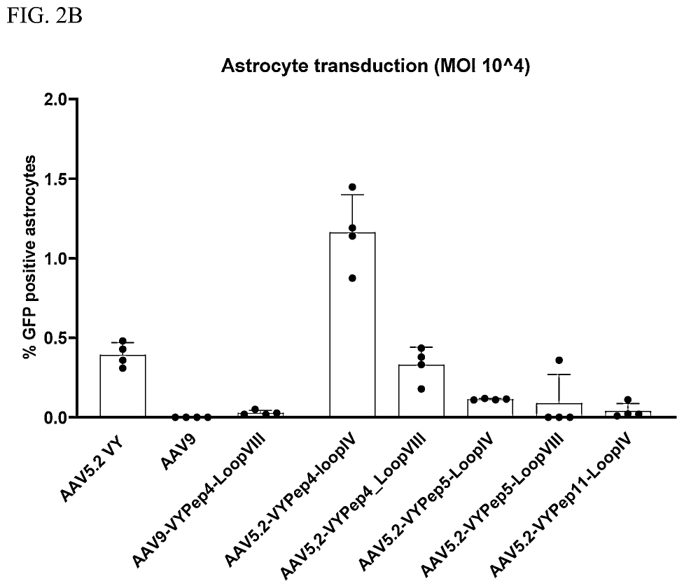

GFP expression from the astrocytes was quantified and expressed as percentage of GFP positive cells out of the total number of cells. The results shown in FIG. 2A and FIG. 2B demonstrate that several of the recombinant AAV capsids successfully transduced human astrocytes at MOIs of 1×10⁴ (FIG. 2B) or 1×10⁶ (FIG. 2A) per cell and that some of the capsids show increased transduction of astrocytes when the peptide is inserted in loop IV compared to loop VIII.

### Example 4: Transduction of Astrocytes Across the Blood Brain Barrier (BBB)

The ability of the recombinant AAV capsids to permeate the blood brain barrier (BBB) was analyzed using a BBB microfluid model.

The BBB model used for testing of the capsids comprises human cell lines of brain endothelial cells (blood side) and astrocytes (brain side) in a three-lane microfluidic platform that harbors 40 chips in a 384-well plate format (OrganoPlate® 3-lane 40). In each chip, a perfused vessel of brain endothelial cells was grown against an extracellular matrix gel, which was patterned by means of surface tension techniques. Astrocytes were added on the other side of the gel to complete the BBB on-a-chip model. Barrier function of the model was studied using fluorescent barrier integrity assays. The different AAV capsids, VYAAV-5.2-GFP, AAV5.2-VYPep4-loop IV-GFP, or AAV5.2-VYPep4-loop VIII-GFP, were added to the chips on the blood side at Day 10 post seeding of the endothelial cells at an MOI of 1×10⁶ per cell. Medium was replaced with fresh human brain microvascular endothelial cell medium and astrocyte medium on the blood side and the brain side, respectively. At Day 6 post transduction with the AAVs, medium was replaced with PBS containing Hoechst 33342, 1:10,000 (Cat. No. 62249, Thermo Fisher Scientific) and the OrganoPlate® was imaged using the ImageXpress PICO at 4× magnification.

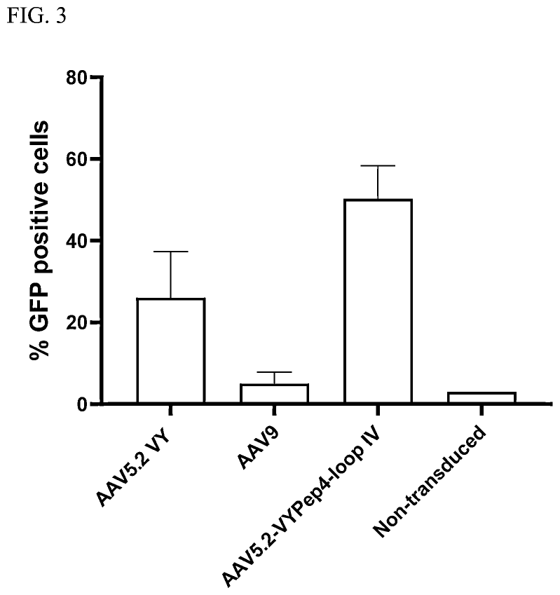

The results in FIG. 3 show that the recombinant AAV capsids successfully permeate the BBB and transduce astrocytes on the brain side of the BBB.

### Example 5: Transduction of Non-Human Primate Astrocytes

The transduction efficiency of the recombinant AAV capsids comprising VYPep4 in loop IV was further analyzed by transducing non-human primate (NHP) primary astrocytes with recombinant capsids.

NHP primary astrocytes were plated at a cell density of 40,000 cells per well of a 24 well plate. Four days after seeding, AAV transduction of the NHP astrocytes was performed at an MOI of 1×10⁵ or 1×10⁶ per cell. Four days post-transduction, the transduced astrocytes were stained with Hoechst and imaged using ImageXpress PICO microscope at 4× magnification.

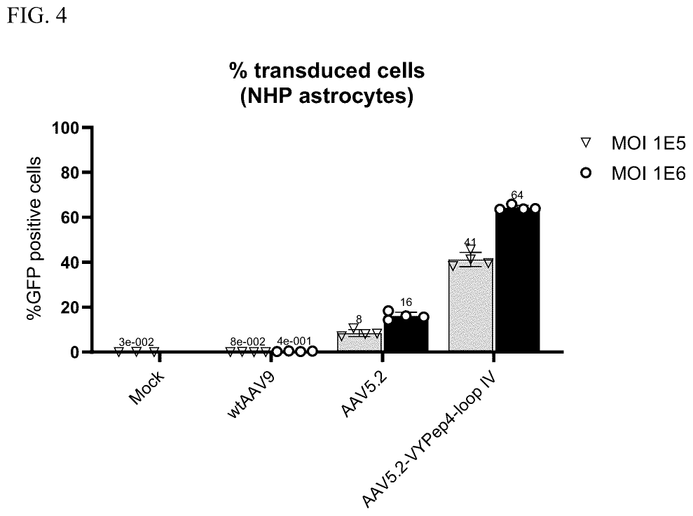

GFP expression from the NHP astrocytes was quantified using the PICO software and expressed as percentage of GFP positive cells out of the total number of cells. The results shown in FIG. 4 demonstrate increased transduction of NHP astrocytes when the peptide is inserted in loop IV compared to AAV5.2 and AAV9 with no peptide inserted.

### Example 6: Transduction of Human Motor Neurons

The transduction efficiency of the recombinant AAV capsids comprising VYPep4 in loop IV was further analyzed by transducing human motor neurons with recombinant capsids.

iPSC-derived human motor neurons were plated at a cell density of 32,000 cells per well of a 96 well plate. AAV transduction was performed at an MOI of 1×10⁵ or 1×10⁶ per cell. One week post-transduction, the transduced motor neurons were fixed with PFA for 20 minutes and stained with an anti-GFP antibody and with Hoechst. After the staining, cells were imaged using the ImageXpress PICO microscope at 10× magnification.

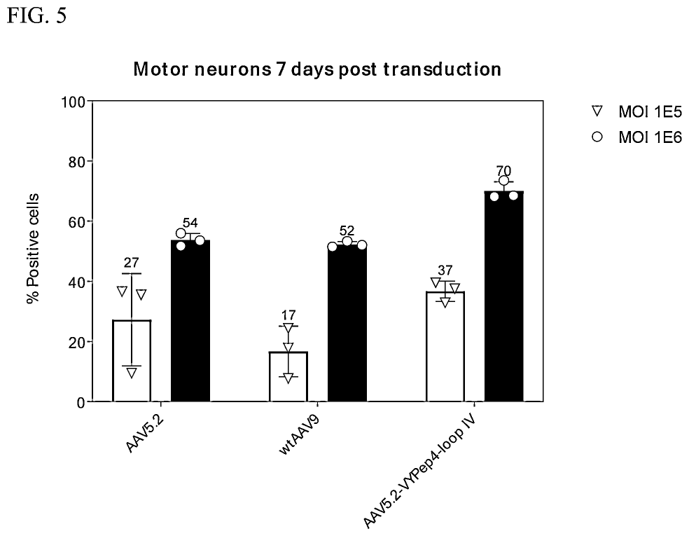

GFP expression from the motor neurons was quantified using the PICO software and expressed as percentage of GFP positive cells out of the total number of cells. The results shown in FIG. 5 demonstrate increased transduction of motor neurons when the peptide is inserted in loop IV compared to AAV5.2 and AAV9 with no peptide inserted.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A recombinant adeno-associated virus (AAV) capsid protein comprising a peptide and a parental capsid protein, wherein the peptide is comprised within loop IV and/or loop VIII of the parental capsid protein, and wherein the peptide comprises an amino acid sequence selected from the group consisting of SEQ ID NOs: 1-6.

2. The recombinant AAV capsid protein of claim 1, wherein the parental capsid protein is a clade A, clade B, clade C, clade D, clade E, clade F, clade G, clade H, clade I, AAVgo.1, AAV3, AAV4, AAV10, AAV11, AAV12, rh.32, rh32.33, rh.33, rh.34, BAAV, or AAV5 capsid protein, or an engineered variant thereof.

3. The recombinant AAV capsid protein of claim 1, wherein the parental capsid protein comprises an amino acid sequence that has at least 95% identity to amino acids 193-725 of SEQ ID NO: 11.

4. The recombinant AAV capsid protein of claim 1, wherein the parental capsid protein comprises an amino acid sequence that has at least 99% identity to amino acids 193-725 of SEQ ID NO: 11.

5. The recombinant AAV capsid protein of claim 1, wherein the parental capsid protein comprises the amino acid sequence of amino acids 193-725 of SEQ ID NO: 11.

6. The recombinant AAV capsid protein of claim 1, wherein the parental capsid protein comprises an amino acid sequence that has at least 95% identity to amino acids 137-725 of SEQ ID NO: 11.

7. The recombinant AAV capsid protein of claim 1, wherein the parental capsid protein comprises an amino acid sequence that has at least 99% identity to amino acids 137-725 of SEQ ID NO: 11.

8. The recombinant AAV capsid protein of claim 1, wherein the parental capsid protein comprises the amino acid sequence of amino acids 137-725 of SEQ ID NO: 11.

9. The recombinant AAV capsid protein of claim 1, wherein the parental capsid protein comprises an amino acid sequence that has at least 95% identity to SEQ ID NO: 10 or 11.

10. The recombinant AAV capsid protein of claim 1, wherein the parental capsid protein comprises an amino acid sequence that has at least 99% identity to SEQ ID NO: 10 or 11.

11. The recombinant AAV capsid protein of claim 1, wherein the parental capsid protein comprises the amino acid of SEQ ID NO: 10 or 11.

12. The recombinant AAV capsid protein of any one of claims 1-11, wherein the peptide is positioned immediately C-terminal to an amino acid in the parental capsid corresponding to amino acid 440, 441, 442, 443, 444, 445, 446, 447, 572, 573, 574, 575, 576, 577, 578, 579, 580, 581, 582, 583, or 584 of SEQ ID NO: 11.

13. The recombinant AAV capsid protein of claim 12, wherein the peptide is positioned immediately C-terminal to an amino acid in the parental capsid corresponding to amino acid 440 of SEQ ID NO: 11.

14. The recombinant AAV capsid protein of claim 12, wherein the peptide is positioned immediately C-terminal to an amino acid in the parental capsid corresponding to amino acid 441 of SEQ ID NO: 11.

15. The recombinant AAV capsid protein of claim 12, wherein the peptide is positioned immediately C-terminal to an amino acid in the parental capsid corresponding to amino acid 442 of SEQ ID NO: 11.

16. The recombinant AAV capsid protein of claim 12, wherein the peptide is positioned immediately C-terminal to an amino acid in the parental capsid corresponding to amino acid 443 of SEQ ID NO: 11.

17. The recombinant AAV capsid protein of claim 12, wherein the peptide is positioned immediately C-terminal to an amino acid in the parental capsid corresponding to amino acid 444 of SEQ ID NO: 11.

18. The recombinant AAV capsid protein of claim 12, wherein the peptide is positioned immediately C-terminal to an amino acid in the parental capsid corresponding to amino acid 445 of SEQ ID NO: 11.

19. The recombinant AAV capsid protein of claim 12, wherein the peptide is positioned immediately C-terminal to an amino acid in the parental capsid corresponding to amino acid 446 of SEQ ID NO: 11.

20. The recombinant AAV capsid protein of claim 12, wherein the peptide is positioned immediately C-terminal to an amino acid in the parental capsid corresponding to amino acid 447 of SEQ ID NO: 11.

21. The recombinant AAV capsid protein of claim 12, wherein the peptide is positioned immediately C-terminal to an amino acid in the parental capsid corresponding to amino acid 572 of SEQ ID NO: 11.

22. The recombinant AAV capsid protein of claim 12, wherein the peptide is positioned immediately C-terminal to an amino acid in the parental capsid corresponding to amino acid 573 of SEQ ID NO: 11.

23. The recombinant AAV capsid protein of claim 12, wherein the peptide is positioned immediately C-terminal to an amino acid in the parental capsid corresponding to amino acid 574 of SEQ ID NO: 11.

24. The recombinant AAV capsid protein of claim 12, wherein the peptide is positioned immediately C-terminal to an amino acid in the parental capsid corresponding to amino acid 575 of SEQ ID NO: 11.

25. The recombinant AAV capsid protein of claim 12, wherein the peptide is positioned immediately C-terminal to an amino acid in the parental capsid corresponding to amino acid 576 of SEQ ID NO: 11.

26. The recombinant AAV capsid protein of claim 12, wherein the peptide is positioned immediately C-terminal to an amino acid in the parental capsid corresponding to amino acid 577 of SEQ ID NO: 11.

27. The recombinant AAV capsid protein of claim 12, wherein the peptide is positioned immediately C-terminal to an amino acid in the parental capsid corresponding to amino acid 578 of SEQ ID NO: 11.

28. The recombinant AAV capsid protein of claim 12, wherein the peptide is positioned immediately C-terminal to an amino acid in the parental capsid corresponding to amino acid 579 of SEQ ID NO: 11.

29. The recombinant AAV capsid protein of claim 12, wherein the peptide is positioned immediately C-terminal to an amino acid in the parental capsid corresponding to amino acid 580 of SEQ ID NO: 11.

30. The recombinant AAV capsid protein of claim 12, wherein the peptide is positioned immediately C-terminal to an amino acid in the parental capsid corresponding to amino acid 581 of SEQ ID NO: 11.

31. The recombinant AAV capsid protein of claim 12, wherein the peptide is positioned immediately C-terminal to an amino acid in the parental capsid corresponding to amino acid 582 of SEQ ID NO: 11.

32. The recombinant AAV capsid protein of claim 12, wherein the peptide is positioned immediately C-terminal to an amino acid in the parental capsid corresponding to amino acid 583 of SEQ ID NO: 11.

33. The recombinant AAV capsid protein of claim 12, wherein the peptide is positioned immediately C-terminal to an amino acid in the parental capsid corresponding to amino acid 584 of SEQ ID NO: 11.

34. The recombinant AAV capsid protein of claim 1, comprising:
(a) an amino acid sequence that has at least 95% sequence identity to amino acids 194-737 of SEQ ID NO: 12, wherein amino acids 445-456 are GGHKAKGPRKLG (SEQ ID NO: 1);
(b) an amino acid sequence that has at least 95% sequence identity to amino acids 194-737 of SEQ ID NO: 13, wherein amino acids 578-589 are GGHKAKGPRKLG (SEQ ID NO: 1);
(c) an amino acid sequence that has at least 95% sequence identity to amino acids 194-734 of SEQ ID NO: 14, wherein amino acids 445-453 are GGHAIYPRH (SEQ ID NO: 2);
(d) an amino acid sequence that has at least 95% sequence identity to amino acids 194-734 of SEQ ID NO: 15, wherein amino acids 578-586 are GGHAIYPRH (SEQ ID NO: 2);
(e) an amino acid sequence that has at least 95% sequence identity to amino acids 194-732 of SEQ ID NO: 16, wherein amino acids 445-451 are RTIGPSV (SEQ ID NO: 3);
(f) an amino acid sequence that has at least 95% sequence identity to amino acids 194-732 of SEQ ID NO: 17, wherein amino acids 578-584 are RTIGPSV (SEQ ID NO: 3);
(g) an amino acid sequence that has at least 95% sequence identity to amino acids 194-732 of SEQ ID NO: 18, wherein amino acids 445-451 are LSSRLDA (SEQ ID NO: 4);
(h) an amino acid sequence that has at least 95% sequence identity to amino acids 194-732 of SEQ ID NO: 19, wherein amino acids 578-584 are LSSRLDA (SEQ ID NO: 4);
(i) an amino acid sequence that has at least 95% sequence identity to amino acids 194-737 of SEQ ID NO: 20, wherein amino acids 445-456 are THRPPMWSPVWP (SEQ ID NO: 5);
(j) an amino acid sequence that has at least 95% sequence identity to amino acids 194-737 of SEQ ID NO: 21, wherein amino acids 578-589 are THRPPMWSPVWP (SEQ ID NO: 5);
(k) an amino acid sequence that has at least 95% sequence identity to amino acids 194-737 of SEQ ID NO: 22, wherein amino acids 445-456 are FGQSSLPRDGPN (SEQ ID NO: 6); or
(l) an amino acid sequence that has at least 95% sequence identity to amino acids 194-737 of SEQ ID NO: 23, wherein amino acids 578-589 are FGQSSLPRDGPN (SEQ ID NO: 6).

35. The recombinant AAV capsid protein of claim 1, comprising:
(a) an amino acid sequence that has at least 99% sequence identity to amino acids 194-737 of SEQ ID NO: 12, wherein amino acids 445-456 are GGHKAKGPRKLG (SEQ ID NO: 1);
(b) an amino acid sequence that has at least 99% sequence identity to amino acids 194-737 of SEQ ID NO: 13, wherein amino acids 578-589 are GGHKAKGPRKLG (SEQ ID NO: 1);
(c) an amino acid sequence that has at least 99% sequence identity to amino acids 194-734 of SEQ ID NO: 14, wherein amino acids 445-453 are GGHAIYPRH (SEQ ID NO: 2);
(d) an amino acid sequence that has at least 99% sequence identity to amino acids 194-734 of SEQ ID NO: 15, wherein amino acids 578-586 are GGHAIYPRH (SEQ ID NO: 2);
(e) an amino acid sequence that has at least 99% sequence identity to amino acids 194-732 of SEQ ID NO: 16, wherein amino acids 445-451 are RTIGPSV (SEQ ID NO: 3);
(f) an amino acid sequence that has at least 99% sequence identity to amino acids 194-732 of SEQ ID NO: 17, wherein amino acids 578-584 are RTIGPSV (SEQ ID NO: 3);
(g) an amino acid sequence that has at least 99% sequence identity to amino acids 194-732 of SEQ ID NO: 18, wherein amino acids 445-451 are LSSRLDA (SEQ ID NO: 4);
(h) an amino acid sequence that has at least 99% sequence identity to amino acids 194-732 of SEQ ID NO: 19, wherein amino acids 578-584 are LSSRLDA (SEQ ID NO: 4);
(i) an amino acid sequence that has at least 99% sequence identity to amino acids 194-737 of SEQ ID NO: 20, wherein amino acids 445-456 are THRPPMWSPVWP (SEQ ID NO: 5);
(j) an amino acid sequence that has at least 99% sequence identity to amino acids 194-737 of SEQ ID NO: 21, wherein amino acids 578-589 are THRPPMWSPVWP (SEQ ID NO: 5);
(k) an amino acid sequence that has at least 99% sequence identity to amino acids 194-737 of SEQ ID NO: 22, wherein amino acids 445-456 are FGQSSLPRDGPN (SEQ ID NO: 6); or
(l) an amino acid sequence that has at least 99% sequence identity to amino acids 194-737 of SEQ ID NO: 23, wherein amino acids 578-589 are FGQSSLPRDGPN (SEQ ID NO: 6).

36. The recombinant AAV capsid protein of claim 1, comprising:
(a) an amino acid sequence that has 100% sequence identity to amino acids 194-737 of SEQ ID NO: 12;
(b) an amino acid sequence that has 100% sequence identity to amino acids 194-737 of SEQ ID NO: 13;
(c) an amino acid sequence that has 100% sequence identity to amino acids 194-734 of SEQ ID NO: 14;
(d) an amino acid sequence that has 100% sequence identity to amino acids 194-734 of SEQ ID NO: 15;
(e) an amino acid sequence that has 100% sequence identity to amino acids 194-732 of SEQ ID NO: 16;
(f) an amino acid sequence that has 100% sequence identity to amino acids 194-732 of SEQ ID NO: 17;
(g) an amino acid sequence that has 100% sequence identity to amino acids 194-732 of SEQ ID NO: 18;
(h) an amino acid sequence that has 100% sequence identity to amino acids 194-732 of SEQ ID NO: 19;
(i) an amino acid sequence that has 100% sequence identity to amino acids 194-737 of SEQ ID NO: 20;
(j) an amino acid sequence that has 100% sequence identity to amino acids 194-737 of SEQ ID NO: 21;
(k) an amino acid sequence that has 100% sequence identity to amino acids 194-737 of SEQ ID NO: 22; or
(l) an amino acid sequence that has 100% sequence identity to amino acids 194-737 of SEQ ID NO: 23.

37. The recombinant AAV capsid protein of claim 1, comprising:
(a) an amino acid sequence that has at least 95% sequence identity to amino acids 138-737 of SEQ ID NO: 12, wherein amino acids 445-456 are GGHKAKGPRKLG (SEQ ID NO: 1);
(b) an amino acid sequence that has at least 95% sequence identity to amino acids 138-737 of SEQ ID NO: 13, wherein amino acids 578-589 are GGHKAKGPRKLG (SEQ ID NO: 1);
(c) an amino acid sequence that has at least 95% sequence identity to amino acids 138-734 of SEQ ID NO: 14, wherein amino acids 445-453 are GGHAIYPRH (SEQ ID NO: 2);
(d) an amino acid sequence that has at least 95% sequence identity to amino acids 138-734 of SEQ ID NO: 15, wherein amino acids 578-586 are GGHAIYPRH (SEQ ID NO: 2);
(e) an amino acid sequence that has at least 95% sequence identity to amino acids 138-732 of SEQ ID NO: 16, wherein amino acids 445-451 are RTIGPSV (SEQ ID NO: 3);
(f) an amino acid sequence that has at least 95% sequence identity to amino acids 138-732 of SEQ ID NO: 17, wherein amino acids 578-584 are RTIGPSV (SEQ ID NO: 3);
(g) an amino acid sequence that has at least 95% sequence identity to amino acids 138-732 of SEQ ID NO: 18, wherein amino acids 445-451 are LSSRLDA (SEQ ID NO: 4);
(h) an amino acid sequence that has at least 95% sequence identity to amino acids 138-732 of SEQ ID NO: 19, wherein amino acids 578-584 are LSSRLDA (SEQ ID NO: 4);
(i) an amino acid sequence that has at least 95% sequence identity to amino acids 138-737 of SEQ ID NO: 20, wherein amino acids 445-456 are THRPPMWSPVWP (SEQ ID NO: 5);
(j) an amino acid sequence that has at least 95% sequence identity to amino acids 138-737 of SEQ ID NO: 21, wherein amino acids 578-589 are THRPPMWSPVWP (SEQ ID NO: 5);
(k) an amino acid sequence that has at least 95% sequence identity to amino acids 138-737 of SEQ ID NO: 22, wherein amino acids 445-456 are FGQSSLPRDGPN (SEQ ID NO: 6); or
(l) an amino acid sequence that has at least 95% sequence identity to amino acids 138-737 of SEQ ID NO: 23, wherein amino acids 578-589 are FGQSSLPRDGPN (SEQ ID NO: 6).

38. The recombinant AAV capsid protein of claim 1, comprising:
(a) an amino acid sequence that has at least 99% sequence identity to amino acids 138-737 of SEQ ID NO: 12, wherein amino acids 445-456 are GGHKAKGPRKLG (SEQ ID NO: 1);
(b) an amino acid sequence that has at least 99% sequence identity to amino acids 138-737 of SEQ ID NO: 13, wherein amino acids 578-589 are GGHKAKGPRKLG (SEQ ID NO: 1);
(c) an amino acid sequence that has at least 99% sequence identity to amino acids 138-734 of SEQ ID NO: 14, wherein amino acids 445-453 are GGHAIYPRH (SEQ ID NO: 2);
(d) an amino acid sequence that has at least 99% sequence identity to amino acids 138-734 of SEQ ID NO: 15, wherein amino acids 578-586 are GGHAIYPRH (SEQ ID NO: 2);
(e) an amino acid sequence that has at least 99% sequence identity to amino acids 138-732 of SEQ ID NO: 16, wherein amino acids 445-451 are RTIGPSV (SEQ ID NO: 3);
(f) an amino acid sequence that has at least 99% sequence identity to amino acids 138-732 of SEQ ID NO: 17, wherein amino acids 578-584 are RTIGPSV (SEQ ID NO: 3);
(g) an amino acid sequence that has at least 99% sequence identity to amino acids 138-732 of SEQ ID NO: 18, wherein amino acids 445-451 are LSSRLDA (SEQ ID NO: 4);
(h) an amino acid sequence that has at least 99% sequence identity to amino acids 138-732 of SEQ ID NO: 19, wherein amino acids 578-584 are LSSRLDA (SEQ ID NO: 4);
(i) an amino acid sequence that has at least 99% sequence identity to amino acids 138-737 of SEQ ID NO: 20, wherein amino acids 445-456 are THRPPMWSPVWP (SEQ ID NO: 5);
(j) an amino acid sequence that has at least 99% sequence identity to amino acids 138-737 of SEQ ID NO: 21, wherein amino acids 578-589 are THRPPMWSPVWP (SEQ ID NO: 5);
(k) an amino acid sequence that has at least 99% sequence identity to amino acids 138-737 of SEQ ID NO: 22, wherein amino acids 445-456 are FGQSSLPRDGPN (SEQ ID NO: 6); or
(l) an amino acid sequence that has at least 99% sequence identity to amino acids 138-737 of SEQ ID NO: 23, wherein amino acids 578-589 are FGQSSLPRDGPN (SEQ ID NO: 6).

39. The recombinant AAV capsid protein of claim 1, comprising:
(a) an amino acid sequence that has 100% sequence identity to amino acids 138-737 of SEQ ID NO: 12;
(b) an amino acid sequence that has 100% sequence identity to amino acids 138-737 of SEQ ID NO: 13;
(c) an amino acid sequence that has 100% sequence identity to amino acids 138-734 of SEQ ID NO: 14;
(d) an amino acid sequence that has 100% sequence identity to amino acids 138-734 of SEQ ID NO: 15;
(e) an amino acid sequence that has 100% sequence identity to amino acids 138-732 of SEQ ID NO: 16;
(f) an amino acid sequence that has 100% sequence identity to amino acids 138-732 of SEQ ID NO: 17;
(g) an amino acid sequence that has 100% sequence identity to amino acids 138-732 of SEQ ID NO: 18;
(h) an amino acid sequence that has 100% sequence identity to amino acids 138-732 of SEQ ID NO: 19;
(i) an amino acid sequence that has 100% sequence identity to amino acids 138-737 of SEQ ID NO: 20;
(j) an amino acid sequence that has 100% sequence identity to amino acids 138-737 of SEQ ID NO: 21;
(k) an amino acid sequence that has 100% sequence identity to amino acids 138-737 of SEQ ID NO: 22; or
(l) an amino acid sequence that has 100% sequence identity to amino acids 138-737 of SEQ ID NO: 23.

40. The recombinant AAV capsid protein of claim 1, comprising:
(a) an amino acid sequence that has at least 95% sequence identity to amino acids 1-737 of SEQ ID NO: 12, wherein amino acids 445-456 are GGHKAKGPRKLG (SEQ ID NO: 1);
(b) an amino acid sequence that has at least 95% sequence identity to amino acids 1-737 of SEQ ID NO: 13, wherein amino acids 578-589 are GGHKAKGPRKLG (SEQ ID NO: 1);
(c) an amino acid sequence that has at least 95% sequence identity to amino acids 1-734 of SEQ ID NO: 14, wherein amino acids 445-453 are GGHAIYPRH (SEQ ID NO: 2);
(d) an amino acid sequence that has at least 95% sequence identity to amino acids 1-734 of SEQ ID NO: 15, wherein amino acids 578-586 are GGHAIYPRH (SEQ ID NO: 2);
(e) an amino acid sequence that has at least 95% sequence identity to amino acids 1-732 of SEQ ID NO: 16, wherein amino acids 445-451 are RTIGPSV (SEQ ID NO: 3);
(f) an amino acid sequence that has at least 95% sequence identity to amino acids 1-732 of SEQ ID NO: 17, wherein amino acids 578-584 are RTIGPSV (SEQ ID NO: 3);
(g) an amino acid sequence that has at least 95% sequence identity to amino acids 1-732 of SEQ ID NO: 18, wherein amino acids 445-451 are LSSRLDA (SEQ ID NO: 4);
(h) an amino acid sequence that has at least 95% sequence identity to amino acids 1-732 of SEQ ID NO: 19, wherein amino acids 578-584 are LSSRLDA (SEQ ID NO: 4);
(i) an amino acid sequence that has at least 95% sequence identity to amino acids 1-737 of SEQ ID NO: 20, wherein amino acids 445-456 are THRPPMWSPVWP (SEQ ID NO: 5);
(j) an amino acid sequence that has at least 95% sequence identity to amino acids 1-737 of SEQ ID NO: 21, wherein amino acids 578-589 are THRPPMWSPVWP (SEQ ID NO: 5);
(k) an amino acid sequence that has at least 95% sequence identity to amino acids 1-737 of SEQ ID NO: 22, wherein amino acids 445-456 are FGQSSLPRDGPN (SEQ ID NO: 6); or
(l) an amino acid sequence that has at least 95% sequence identity to amino acids 1-737 of SEQ ID NO: 23, wherein amino acids 578-589 are FGQSSLPRDGPN (SEQ ID NO: 6).

41. The recombinant AAV capsid protein of claim 1, comprising:
(a) an amino acid sequence that has at least 99% sequence identity to amino acids 1-737 of SEQ ID NO: 12, wherein amino acids 445-456 are GGHKAKGPRKLG (SEQ ID NO: 1);
(b) an amino acid sequence that has at least 99% sequence identity to amino acids 1-737 of SEQ ID NO: 13, wherein amino acids 578-589 are GGHKAKGPRKLG (SEQ ID NO: 1);
(c) an amino acid sequence that has at least 99% sequence identity to amino acids 1-734 of SEQ ID NO: 14, wherein amino acids 445-453 are GGHAIYPRH (SEQ ID NO: 2);
(d) an amino acid sequence that has at least 99% sequence identity to amino acids 1-734 of SEQ ID NO: 15, wherein amino acids 578-586 are GGHAIYPRH (SEQ ID NO: 2);
(e) an amino acid sequence that has at least 99% sequence identity to amino acids 1-732 of SEQ ID NO: 16, wherein amino acids 445-451 are RTIGPSV (SEQ ID NO: 3);
(f) an amino acid sequence that has at least 99% sequence identity to amino acids 1-732 of SEQ ID NO: 17, wherein amino acids 578-584 are RTIGPSV (SEQ ID NO: 3);
(g) an amino acid sequence that has at least 99% sequence identity to amino acids 1-732 of SEQ ID NO: 18, wherein amino acids 445-451 are LSSRLDA (SEQ ID NO: 4);
(h) an amino acid sequence that has at least 99% sequence identity to amino acids 1-732 of SEQ ID NO: 19, wherein amino acids 578-584 are LSSRLDA (SEQ ID NO: 4);
(i) an amino acid sequence that has at least 99% sequence identity to amino acids 1-737 of SEQ ID NO: 20, wherein amino acids 445-456 are THRPPMWSPVWP (SEQ ID NO: 5);
(j) an amino acid sequence that has at least 99% sequence identity to amino acids 1-737 of SEQ ID NO: 21, wherein amino acids 578-589 are THRPPMWSPVWP (SEQ ID NO: 5);
(k) an amino acid sequence that has at least 99% sequence identity to amino acids 1-737 of SEQ ID NO: 22, wherein amino acids 445-456 are FGQSSLPRDGPN (SEQ ID NO: 6); or
(l) an amino acid sequence that has at least 99% sequence identity to amino acids 1-737 of SEQ ID NO: 23, wherein amino acids 578-589 are FGQSSLPRDGPN (SEQ ID NO: 6).

42. The recombinant AAV capsid protein of claim 1, comprising an amino acid sequence selected from the group consisting of SEQ ID NOs: 12-23.

43. The recombinant AAV capsid protein of claim 1, wherein the amino acid sequence of the recombinant AAV capsid protein consists of an amino acid sequence selected from the group consisting of SEQ ID NOs: 12-23.

44. An isolated polynucleotide encoding the recombinant AAV capsid protein of any one of claims 1-43.

45. A vector comprising the polynucleotide of claim 44.

46. The vector of claim 45, which is a plasmid or a viral vector.

47. The vector of claim 46, wherein the viral vector is a retrovirus vector, a herpes virus vector, a baculovirus vector, or an adenovirus vector.

48. The vector of any one of claims 45-47, which is an expression vector.

49. A recombinant cell comprising the polynucleotide of claim 44 or the vector of any one of claims 45-47.

50. A method of producing a recombinant AAV capsid protein, the method comprising culturing the recombinant cell of claim 49 under conditions where the polynucleotide is expressed, and the capsid protein is produced.

51. A recombinant adeno-associated virus (rAAV) comprising:
(a) a capsid comprising one or more of the recombinant AAV capsid protein of any one of claims 1-43; and
(b) an rAAV genome.

52. The rAAV of claim 51, wherein the rAAV genome comprises a transgene.

53. The rAAV of claim 52, wherein the transgene encodes a polypeptide or a non-coding RNA.

54. The rAAV of claim 52 or 53, wherein the transgene encodes an antibody or a fragment thereof.

55. The rAAV of claim 54, wherein the transgene encodes an scFv, a nanobody, or a VHH.

56. A method of delivering a transgene to a cell, the method comprising contacting the cell with the rAAV of any one of claims 51-55, under conditions where the cell is transduced, and the transgene is expressed.

57. The method of claim 56, wherein the cell is a muscle cell, microglia, astrocyte, neuron, blood brain barrier endothelial cell (BBB EC), pericyte, oligodendrocyte, ependymal cell, or cardiomyocyte.

58. The method of claim 57, wherein the neuron is a pyramidal neuron, rosehip neuron, or purkinje cell.

59. A method of delivering a transgene to muscle tissue in a subject in need thereof, the method comprising administering the rAAV of any one of claims 51-55 to the subject.

60. The method of claim 59, wherein the subject has a disease associated with muscle tissue.

61. A method of treating a neuromuscular or neurodegenerative disease or disorder in a subject in need thereof, comprising administering an effective amount of an rAAV of any one of claims 51-55 to the subject.

62. The method of claim 61, wherein the neuromuscular disease or disorder is selected from the group consisting of myopathy, hereditary cardiomyopathy, metabolic myopathy, distal myopathy, muscular dystrophy, congenital myopathy, spinal muscular atrophy (SMA), motor neuron disease, congenital myopathy, congenital muscular dystrophy, motor neuron disease, Duchenne muscular dystrophy, Becker muscular dystrophy, limb-girdle muscular dystrophies, myotonic dystrophy, myotubular myopathy, centronuclear myopathy, nemaline myopathy, selenoprotein N-related myopathy, Pompe disease, glycogen storage disease III, spinal muscular atrophy, amyotrophic lateral sclerosis (ALS), Charcot-Marie-Tooth disease, multiple sclerosis, myositis, polymyositis, and dermatomyositis.

63. The method of claim 61, wherein the neurodegenerative disease or disorder is selected from the group consisting of motor neuron disease (MND), parkinsonism syndrome, Alzheimer's dementia, progressive supranuclear palsy (PSP), Huntington's disease, multiple system atrophy (MSA), spinocerebellar ataxia (SCA), and Friedreich's ataxia.

64. The method of any one of claims 59-63, wherein the rAAV is administered to the subject intravenously, intraperitoneally, subcutaneously, intramuscularly, intrathecally, or intradermally.
